(19) United States
(12) Patent Application Publication
(10) Pub. No.: US 2023/0384251 A1
(43) Pub. Date: Nov. 30, 2023

(54) Methods and Systems of Fabricating Electrical Devices by Micro-Molding

(71) Applicant: Mesoline Inc., Charlotte, NC (US)

(72) Inventors: Marnix P. Rebergen, Rotterdam (NL); Michael D. Kelzenberg, Glendale, CA (US); Thomas C.R. Russell, Amsterdam (NL); Serhii Mytnyk, Capelle aan den Ijssel (NL)

(73) Assignee: Mesoline Inc., Charlotte, NC (US)

(21) Appl. No.: 18/448,044

(22) Filed: Aug. 10, 2023

Related U.S. Application Data
(63) Continuation of application No. 17/342,338, filed on Jun. 8, 2021.
(60) Provisional application No. 63/036,357, filed on Jun. 8, 2020.
(60) Provisional application No. 63/086,367, filed on Oct. 1, 2020.

(51) Int. Cl. G01N 27/12; G01N 33/00; B22F 7/08; B22F 3/00; B41M 3/00; B41M 7/00; B22F 3/105

(52) U.S. Cl. CPC G01N 27/128; G01N 27/127; G01N 33/0027; B22F 7/08; B22F 3/004; B41M 3/006; B41M 7/0081; B22F 3/105; B22F 2304/05

(57) ABSTRACT

Systems of electrical devices with high-resolution components and methods of fabricating the electrical devices using micro-molding processes are described. Small foot print electrical devices can be achieved by fabricating components with highly conductive materials, and with closely spaced components.

### CROSS-REFERENCE TO RELATED APPLICATIONS

The current application is a continuation of U.S. patent application Ser. No. 17/342,338 entitled “Methods and Systems of Fabricating Electrical Devices by Micro-Molding” filed Jun. 8, 2021, which claims the benefit of priority under 35 U.S.C. § 119(e) to U.S. Provisional Patent Application No. 63/036,357 entitled “Small-Footprint Antenna Structure with High-Aspect-Ratio Conductors” filed Jun. 8, 2020, and to U.S. Provisional Patent Application No. 63/086,367 entitled “Micro-Molded Gas Sensor” filed Oct. 1, 2020. The disclosures of which are hereby incorporated by references in their entirety for all purposes.

### FIELD OF THE INVENTION

The present invention generally relates to methods and systems of fabricating electrical devices by micro-molding; and more particularly to methods and systems of fabricating electrical devices having high resolution features using micro-molding processes.

### BACKGROUND

Micro-molding is a manufacturing process that can produce small and high-precision parts and components with micron tolerances. The process can start by creating a mold that has a cavity in the shape of the part desired. Thermoplastic or resin can be rapidly injected into the cavity, creating the part or component at high speed. Materials such as polyether ether ketone (PEEK), polyetherimide (PEI), carbon filled liquid crystal polymer (LCP) or glass filled nylons can be used in micro-molding processes. Soft durometer or elastomeric resins can also be applied.

### BRIEF SUMMARY

Systems and methods in accordance with various embodiments of the invention enable the design and fabrication of electrical devices including (but not limited to) gas sensors, antennas, and inductors using micro-molding processes. Many embodiments provide design and structures of micro-molding machines used in micro-molding processes. Micro-molding machines in accordance with several embodiments can fabricate high resolution electrical conductors with high-aspect-ratio. Many embodiments utilize high-aspect-ratio components to produce compact, high-performance electrical devices in various configurations. Several embodiments provide fabrication methods of high resolution and/or high-aspect-ratio components at a low cost. Some embodiments provide that micro-molding processes provide consistent, repeatable, and simplified manufacturing processes.

The gas sensors and/or gas sensor elements fabricated with micro-molding processes in accordance with several embodiments have lower power consumption, increased sensitivity, improved selectivity, increased consistency and controllability, and reduced footprint. Many embodiments provide micro-molding fabrication of small-footprint antennas including (but not limited to) near-field antennas, or far-field antennas. Several embodiments provide compact antenna coil structures with high inductance and low series resistance for a given antenna footprint and conductor length. The high inductance and low series resistance of antenna structures can be achieved by fabricating antenna coils with highly conductive materials, and with closely spaced and high aspect-ratio electrical conductors (traces) in accordance with certain embodiments. Several embodiments provide that electrically conductive components of the electrical devices can be made from nanoparticles including (but not limited to) metallic nanoparticles.

One embodiment of the invention includes a micro-molded gas sensor, comprising at least one gas-sensor element, wherein the at least one gas-sensor element comprising a nano-porous electrical conductor, where the nano-porous electrical conductor comprising fused nanoparticles; at least one first electrode electrically connected to a first end of the at least one gas-sensor element; and at least one second electrode electrically connected to a second end of the at least one gas-sensor element; where the at least one gas-sensor element has a corresponding first electrode and second electrode pair, and an electrical characteristic of the at least one gas-sensor element measured by the at least one first electrode and the at least one second electrode changes in response to an ambient gas in contact with the nano-porous electrical conductor.

In another embodiment, the micro-molded gas sensor further comprising a first gas-sensor element and a second gas-sensor element, where the first gas-sensor element comprises a first nanoparticle composition, and the second gas-sensor element comprises a second nanoparticle composition different from the first nanoparticle composition.

In a further embodiment, the micro-molded gas sensor further comprising a first gas-sensor element and a second gas-sensor element, where the first gas-sensor element has a first form factor, and the second gas-sensor element has a second form factor different from the first form factor.

In still another embodiment, the micro-molded gas sensor further comprising a micro-heater to heat the at least one gas-sensor element.

In a yet further embodiment, the micro-heater comprises a plurality of micro-heater segments that are individually controllable to provide a different temperature in each of the plurality of micro-heater segments simultaneously.

In a still further embodiment, the micro-molded gas sensor also includes a sensor controller electrically connected to the at least one first electrode and electrically connected to the at least one second electrode, wherein the sensor controller is operable to provide electrical current to, and measure the resistivity of, the at least one gas-sensor element.

In yet another embodiment, the micro-molded gas sensor further comprising a substrate; a micro-heater disposed on the substrate; and an electrically insulating layer disposed on the micro-heater, where the at least one first electrode and the at least one second electrode are disposed on the electrically insulating layer and the at least one gas-sensor element is disposed on the corresponding first electrode and second electrode pair.

In a further embodiment again, the at least one gas-sensor element does not extend beyond the micro-heater.

In a still yet further embodiment, the substrate incorporates at least one membrane, wherein the membrane has a thickness less than about 1 micron.

In another additional embodiment, the nanoparticles are selected from the group consisting of metal nanoparticles, metal-oxide nanoparticles, and doped metal-oxide nanoparticles.

In another embodiment again, the metal-oxide nanoparticles are one or more of: SnO₂, TiO₂, WO₃, ZnO, In₂O₃, Cd:ZnO, CrO₃, and V₂O₅.

In a yet further embodiment again, the metal-oxide nanoparticles are doped with Al, Pt, Pd, Au, Ag, Ti, Cu, Fe, Sb, Mo, Ce, Mn, Rh₂O₃, or carbon nanotubes.

In a still yet further embodiment, the at least one gas-sensor element has a height in the range of about 1 μm to about 20 μm, and a width in the range of about 1 μm to about 50 μm.

In still yet another embodiment, the at least one gas-sensor element has a surface roughness of less than about 100 nm RMS.

In a further embodiment again, the ratio between an element height of the at least one gas-sensor element and an element width of the at least one gas-sensor element is no less than 2.

In still yet another embodiment, the ratio between an element height of the at least one gas-sensor element and an element width of the at least one gas-sensor element is no greater than 0.5.

In a still further embodiment again, the ratio between a spacing between at least two adjacent gas sensor elements and an element width of the at least one gas-sensor element is no more than 4.

In a still further additional embodiment, the micro-molded gas sensor further comprising at least one force electrode that injects current or voltage into the at least one gas-sensor element, and at least one sense electrode that measures a change in an electrical characteristic.

Still another additional embodiment includes a micro-molding machine, comprising a stamp having a first channel disposed on a surface of the stamp and a second channel disposed on the surface of the stamp; a first inlet port connected to the first channel and a second inlet port separate from the first inlet port connected to the second channel; a first nanoparticle ink supply for supplying a first nanoparticle ink to the first inlet port and a second nanoparticle ink supply separate from the first nanoparticle ink supply for supplying a second nanoparticle ink to the second inlet port, where the first nanoparticle ink comprises a first nanoparticle composition and the second nanoparticle ink comprises a second nanoparticle composition different from the first nanoparticle composition; a pump or a dispenser for pumping or dispensing the first nanoparticle ink through the first inlet port and the first channel and for pumping or dispensing the second nanoparticle ink through the second inlet port and the second channel; and a contact mechanism for contacting the surface of the stamp to a substrate.

In another additional embodiment, the first channel has a first form factor and the second channel has a second form factor different from the first form factor.

A yet further embodiment again includes an outlet port connected to the first or second channels, wherein the pump or dispenser is operable to provide a pressure less than an atmospheric pressure to the outlet port.

Another further embodiment again includes a method of micro-molding a gas-sensor element, comprising:
providing a substrate having a substrate surface;
providing a stamp comprising a mold layer having a support side and a channel side and a support layer disposed in contact with the support side, wherein the mold layer comprises (i) a first channel having a first form factor disposed on the channel side, a first inlet port connected to the first channel, and a first outlet port connected to the first channel; and (ii) a second channel having a second form factor disposed on the channel side, a second inlet port connected to the second channel, and a second outlet port connected to the second channel;
providing a first nanoparticle ink comprising a first nanoparticle composition and a second nanoparticle ink comprising a second nanoparticle composition;
disposing the mold layer in contact with the substrate surface;
pumping or dispensing the first nanoparticle ink through the first inlet port and into the first channel and pumping or dispensing the second nanoparticle ink through the second inlet port and into the second channel;
curing the first nanoparticle ink in the first channel to form a first nano-porous fused nanoparticle electrical conductor having an electrical conductivity that changes in response to a first ambient gas in contact with the first nano-porous fused nano-particle electrical conductor;
curing the second nanoparticle ink in the second channel to form a second nano-porous fused nanoparticle electrical conductor having an electrical conductivity that changes in response to a second ambient gas in contact with the second nano-porous fused nanoparticle electrical conductor; and
removing the stamp to form a free-standing gas-sensor element on the substrate surface.

In yet another embodiment again, the first nanoparticle composition is different from the second nanoparticle composition, and the first form factor is the same as the second form factor.

In still another further embodiment, the first nanoparticle composition is the same as the second nanoparticle composition, and the first form factor is different from the second form factor.

In another further additional embodiment, the first nanoparticle composition is different from the second nanoparticle composition, and the first form factor is different from the second form factor.

In still yet another embodiment, the support layer is more rigid than the mold layer.

In still another additional embodiment, the channel has a height in a direction into the mold layer from the channel side, and the height is greater than a width of the channel on the channel side.

A further embodiment again includes heating the nanoparticle ink or exposing the nanoparticle ink to an electromagnetic radiation to accelerate the curing step.

A still further embodiment includes sintering the nanoparticles by heating the nanoparticles or by exposing the nanoparticles to an electromagnetic radiation.

Still another additional embodiment includes providing an inlet pressure to the inlet port and an outlet pressure to the outlet port during the pumping of dispensing step, wherein the inlet pressure is greater than the outlet pressure.

In a yet further embodiment, the step of pumping or dispensing the nanoparticle ink causes the nanoparticle ink to flow through the channel, wherein the flow of nanoparticle ink is driven at least in part by a capillary pressure in the channel.

In yet another embodiment again, the step of pumping or dispensing the nanoparticle ink causes the nanoparticle ink to flow through the channel, wherein the flow of nanoparticle ink is driven by applying a pressure to the inlet port or applying a vacuum to the outlet port.

In still another further embodiment, the stamp comprises a material selected from the group consisting of polydimethylsiloxane, polymethyl methacrylate, and polyurethane.

In another further additional embodiment, at least one ink reservoir is incorporated into the stamp.

In still yet another embodiment, the mold layer is reinforced by incorporation of nanoparticles, or by inclusion of a fiber mesh comprising a material selected from the group consisting of glass, steel, carbon, and nylon.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention.

FIG. 1 illustrates a plan view of gas-sensor elements in accordance with an embodiment of the invention.

FIG. 2 illustrates a cross section view of gas-sensor elements in accordance with an embodiment of the invention.

FIG. 3 illustrates a plan view of different gas-sensor elements in accordance with an embodiment of the invention.

FIG. 4 illustrates a cross section view of different gas-sensor elements in accordance with an embodiment of the invention.

FIG. 5 illustrates a plan view of micro-heater segments incorporated in gas-sensors in accordance with an embodiment of the invention.

FIG. 6 illustrates a perspective and cross section of a detail inset of gas-sensor elements in accordance with an embodiment of the invention.

FIG. 7 illustrates a gas sensor with different substrate thickness in accordance with an embodiment of the invention.

FIG. 8 illustrates a plan view of a gas sensor incorporating multiple gas-sensor elements in accordance with an embodiment of the invention.

FIGS. 9A-9D illustrate a plan view and cross sections of micro-molding machines in accordance with an embodiment of the invention.

FIG. 10 illustrates a process of micro-molding fabrication process in accordance with an embodiment of the invention.

FIG. 11A-11D illustrate successive cross section views of sequential structures during a micro-molding process of fabricating a gas-sensor in accordance with an embodiment of the invention.

FIG. 12A illustrates a plan view of a high-aspect-ratio antenna in accordance with an embodiment of the invention.

FIG. 12B illustrates a cross section view of a high-aspect-ratio antenna in accordance with an embodiment of the invention.

FIG. 13A illustrates a plan view of a coil antenna in accordance with an embodiment of the invention.

FIG. 13B illustrates a cross section view of a coil antenna in accordance with an embodiment of the invention.

FIG. 14A illustrates a plan view of an antenna with antenna length L in accordance with an embodiment of the invention.

FIG. 14B illustrates a cross section view of an antenna with antenna length L in accordance with an embodiment of the invention.

FIG. 15 illustrates a plan view of a coil antenna incorporating thermal strain reliefs in accordance with an embodiment of the invention.

FIG. 16A illustrates a plan view of a micro-mold stamp in accordance with an embodiment of the invention.

FIGS. 16B-16C illustrate cross section views of a micro-mold stamp in accordance with an embodiment of the invention.

FIGS. 17A-17D illustrate successive cross section views of sequential structures during a micro-molding process of fabricating a high-aspect-ratio antenna in accordance with an embodiment of the invention.

FIG. 18 illustrates a cross section view of an antenna system in accordance with an embodiment of the invention.

FIG. 19 illustrates an exploded perspective view of a multi-layer high-aspect-ratio antenna in accordance with an embodiment of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. The figures are not drawn to scale since the variation in size of various elements in the Figures is too great to permit depiction to scale.

### DETAILED DESCRIPTION

Turning now to drawings, systems and methods for fabricating electrical devices using micro-molding processes are described. Many embodiments provide design and structures of micro-molding machines used in micro-molding processes. Micro-molding machines in accordance with several embodiments can fabricate high resolution electrical conductors with high-aspect-ratio. The electrical conductors can be integrated in electrical devices including (but not limited to) gas sensors, inductors, antennas. Many embodiments provide that micro-molding machines include at least a stamp. At least one ink supply can be supplied to the stamp during micro-molding processes. Multiple ink supplies in accordance with several embodiments can supply same and/or different inks to the micro-molding stamp. In several embodiments, micro-molding machines have channels of same and/or different form factors.

Many embodiments provide micro-molding processes of making high-aspect-ratio electrical components and/or devices. The gas sensors and/or gas sensor elements fabricated with micro-molding processes in accordance with several embodiments have lower power consumption, increased sensitivity, improved selectivity, increased consistency and controllability, and reduced footprint. Some embodiments provide that micro-molding processes provide consistent, repeatable, and simplified manufacturing processes. In certain embodiments, gas sensors can include at least one gas sensor element. Many embodiments provide that the multiple gas-sensor elements can comprise same or different materials, and/or have same or different form factors. In some embodiments, the gas-sensor elements can be disposed on sensing electrodes over at least one micro-heater. Several embodiments provide that the gas-sensor elements can be exposed to an ambient gas. In certain embodiments, the micro-heaters can heat the gas-sensor elements. A number of embodiments provide that the sensing electrodes can measure the electrical characteristics of the gas-sensor elements. The gas-sensor elements can be nano-porous electrical conductors made of (but not limited to) fused nanoparticles. Electrical characteristics of gas-sensor elements in accordance with many embodiments can change in response to an ambient gas in contact with the nano-porous electrical conductors. The electrical characteristics can include (but are not limited to) resistivity, capacitance, inductance, phase, and any combinations thereof.

In many embodiments, micro-heaters can provide heat to gas-sensor elements to control the temperature of gas-sensor elements. Heat can decrease the resistivity of the gas sensor elements and enhance the interaction of target gas molecules with the sensing material and can therefore increase the sensitivity of the sensor elements to the target gas. In many embodiments, micro-heaters of gas-sensors can include individually controllable micro-heater segments. The individually controllable micro-heater segments in accordance with several embodiments can be individually controllable to provide a different temperature in each micro-heater segment simultaneously, enabling better selectivity of each sensing element towards its target gas. Some embodiments provide that each micro-heater segment can be associated with and/or in thermal contact with a different gas-sensor element. In such embodiments, the multiple micro-heater segments can simultaneously heat corresponding gas-sensor elements to different temperatures. Gas-sensor elements heated to different temperatures in accordance with many embodiments can be applied to detect different gases and/or different concentrations of gases, through individual and separate micro-heater electrodes. By providing differently controllable and different gas-sensor elements, gas sensors can measure different gases and/or different gas concentrations at the same time and can be used as sensing devices including (but not limited to) electronic noses.

Decreasing the footprint of gas sensor elements can decrease a total area of the microheater in accordance with many embodiments without compromising the temperature uniformity of gas-sensor elements. Microheater power draw may increase with area, thus the decrease of the total area of the microheater can result in a decrease in gas sensor power consumption, facilitating the use of gas sensors in accordance with several embodiments in battery-powered electronics.

Many embodiments provide that gas-sensor elements of gas-sensors can have geometric shapes including (but not limited to) linear and straight line, curved, or spiral. Gas-sensor elements can have different cross sections including (but not limited to) square, rectangular, cubic, circular, or cylindrical. In several embodiments, gas-sensor element height H can be greater than element width W. In certain embodiments, gas-sensor element height H can be smaller than element width W.

Multiple different gas-sensor elements in accordance with many embodiments can comprise electrical conductors made of same or different nanoparticles. Different nanoparticle compositions of multiple different gas-sensor elements in accordance with certain embodiments can be sensitive to different gases and/or gas concentrations. The different nanoparticles in accordance with several embodiments can include (but are not limited to) different nanoparticle materials, different nanoparticle doping, different nanoparticle sizes, and any combinations thereof. The nano-porous electrical conductors of different gas-sensor elements in accordance with some embodiments can have different nano-porosities including (but not limited to) nanopore sizes, and quantities of nano-pores in the nano-porous electrical conductors. Certain embodiments provide that nanoparticles of gas-sensor elements can have diameters ranging from about 1 nm to about 1 micron.

Many embodiments provide that nanoparticles of gas sensors can include (but are not limited to) metal nanoparticles, metal-oxide nanoparticles, or doped metal-oxide nanoparticles. Metal-oxide nanoparticles in accordance with certain embodiments can include (but are not limited to) SnO₂, TiO₂, ITO, CdSe, WO₃, ZnO, In₂O₃, Cd:ZnO, CrO₃, V₂O₅, and any combinations thereof. In some embodiments, metal-oxide nanoparticles can be doped with Al, Pt, Pd, Au, Ag, Ti, Cu, Fe, Sb, Mo, Ce, Mn, Rh₂O₃, or carbon nanotubes (CNTs) to improve the selectivity of the sensor. In several embodiments, assemblies of nanoparticles can comprise materials including (but not limited to) non-conductive materials, and/or dielectric materials. The non-conductive materials in accordance with embodiments can be sensitive to gases and affect the response of conductive materials in the nano-porous electrical conductor, and/or can be useful for constructing the nano-porous electrical conductor. In a number of embodiments, nanoparticle ink can be provided as a suspension in liquid solvent including (but not limited to) aqueous dispersants, and/or organic solvents. Nanoparticles in accordance with several embodiments can have viscosities in a range from about 0.3 centipoise to about 300 centipoises. In some embodiments, nanoparticles comprise different nanoparticles made of different conductive or non-conductive materials and can be distributed isotropically or anisotropically in gas-sensor elements.

Many embodiments provide that substrates of gas-sensors can include (but are not limited to) glass, polymers, semiconductors, ceramics, quartz, metals, paper, and/or sapphire. In several embodiments, the substrates for gas-sensors can be a printed-circuit board (PCB) substrate, or liquid-crystal polymer (LCP) materials. Some embodiments provide that the substrates can be rigid, flexible, and/or substantially planar. In a number of embodiments, the substrates can be found in the display, integrated circuit, electronics assembly, or circuit board industries. In some embodiments, the substrates may contain CMOS and/or MEMS devices, integrated circuits, microprocessors, microcontrollers, angle measurement circuitry, RF circuits, and transceivers.

Many embodiments provide high-aspect-ratio antennas including (but not limited to) inductors fabricated using micro-molding processes. Many embodiments provide the high-aspect-ratio antennas comprise inductive coils. Inductive coils in accordance with some embodiments have helical and/or spiral arrangement of conductive electrical material. In several embodiments, the electrical conductors have high-aspect-ratios. In many embodiments, antennas with high-aspect-ratio conductors increase the cross-sectional area of the conductor, for a given conductor width, thus reducing the electrical resistance of antennas. Several embodiments provide that the antenna footprint can be greatly reduced.

In many embodiments, high-aspect-ratio electrical conductors are arranged in a variety of configurations for high-performance inductors and antennas including (but not limited to) near-field antennas. Certain embodiments provide that the electrical wires and/or traces can be arranged in configurations including (but not limited to) planar rectangular, circular, and/or hexagonal spiral on a substrate to form a coil. The antenna in accordance with many embodiments can have a cross section including (but not limited to) rectangular, triangular, quadrilateral, or with a curved surface. In some embodiments, coils can be extended into the normal direction with respect to the substrate, so that the conductor has an increased aspect ratio. The antenna can be electrically connected to a circuit that operates or responds to antenna. Many embodiments provide high-aspect-ratio antennas can be integrated into an electronic circuit including (but not limited to) a tuned antenna system. In several embodiments, components including (but not limited to) circuits, integrated circuits (ICs), resistors, and capacitors can be incorporated into the antenna systems. The added components in accordance with some embodiments can be placed inside and/or outside the coil. In certain embodiments, the components can be placed within a different circuit plane.

In some embodiments, several coils can be stacked to increase the inductance of the coil. In many embodiments, high-aspect-ratio antennas can be a multi-layer antenna. Each antenna layer in accordance with some embodiments can be separated by an insulator from adjacent layers and connected through electrical vias. In some embodiments, the inductance of the multi-layer coil structure can be improved compared to a single-layer coil. The coils in accordance with some embodiments can be located on a same plane and/or substrate. In several embodiments, the coils can be placed at subsequent planes and/or substrates along the same axis. The designs of the coil can be symmetrical and/or asymmetrical in accordance with certain embodiments.

In many embodiments, antenna coils with high-aspect-ratios have small-footprint and exhibit high inductance and low series resistance. Several embodiments provide that the high inductance and low series resistance of antenna structures can be achieved by fabricating antenna coils with highly conductive materials, and with closely spaced and high aspect-ratio traces. Several embodiments provide that electrically conductive traces of the high-aspect-ratio antennas can be made from particles including (but not limited to) electrically conductive particles, metallic nanoparticles, electrically non-conductive (dielectric) particles, and semi-conducting particles. In some embodiments, particles comprise nanoparticles made of different conductive and/or non-conductive materials. In several embodiments, nanoparticles can be distributed isotropically and/or anisotropically in antenna. Examples of metallic nanoparticles include (but are not limited to): silver, copper, gold, nickel, and any combinations thereof. Examples of semi-conducting particles include (but are not limited to) metal oxide particles. Many embodiments provide that the particles can be provided as a suspension in a liquid solvent. Nanoparticles in accordance with several embodiments can have diameters in the range from about 1 nm to about 5 μm.

Many embodiments provide that a high-aspect-ratio antenna structure can include a plurality of antennas including (but not limited to) coil antennas disposed on a substrate. Several embodiments provide high-aspect-ratio antennas can be constructed using a micro-mold stamp. In many embodiments, the high-aspect-ratio conductors can be constructed from nanoparticle inks cured in channels disposed in micro-mold stamps applied onto a substrate surface. This process enables antennas and inductors to be made with dimensions suitable for small and portable electronic devices. Several embodiments provide that antennas and conductors have dimensions in the range from about 1 μm to about 100 μm. In certain embodiments, antennas have an aspect ratio (a ratio of conductor height to conductor width) of greater than 1.

Many embodiments provide micro-molding processes to fabricate high-aspect-ratio antennas. Several embodiments incorporate micro-molding machines including micro-molding stamps in fabricating the antennas. In certain embodiments, micro-molding stamps can print high-aspect-ratio conductors comprising nanoparticles on a substrate to form high-aspect-ratio antennas. Many embodiments provide that the well-controlled surface roughness of micro-molding stamps and small sizes of nanoparticles ink enable a much smaller root-mean-square surface roughness of high-aspect-ratio than the skin depth of the conductor. In several embodiments, signals produced in antennas have reduced signal attenuation at high frequencies (between 1 MHz and 1 THz). At high frequencies (frequencies greater than 1 MHz) the skin effect can become significant. For example, in the UHF band the skin depth comprises several microns and the vast majority of the electric current can flow within a distance of about 5 times the skin depth of the surfaces of the conductor. Surface roughness of the conductors thus may lead to measurable changes in resistance, which in turn leads to an increase in signal attenuation. Generally, the root-mean-square surface roughness should be much smaller than the skin depth of the electric field in the conductor to avoid additional attenuation of the signal. Some embodiments provide that micro-molding stamps have well controlled surface roughness and nanoparticle have small sizes. The printed electrical conductors of high-aspect-ratio antennas in accordance with many embodiments have a greatly decreased root-mean-square surface roughness compared to conventional manufacturing methods including (but not limited to) screen-printing and inkjet-printing. Several embodiments provide that the surface roughness of antennas can be much below the skin depth of the conductor, and signals produced in antennas have reduced signal attenuation at high frequencies (frequencies between about 1 MHz and about 1 THz).

Many embodiments provide micro-molding methods of manufacturing the high-aspect-ratio antennas and/or coils at reasonable costs. In several embodiment, antennas with high-aspect-ratio conductors can be fabricated as free-standing structures formed or deposited on a substrate. Some embodiments provide that the substrate can be a printed-circuit board (PCB) substrate. Substrates can be found in the display, integrated circuit, electronics assembly, or circuit board industries in accordance with certain embodiments. In some embodiments, the substrate may contain CMOS and/or MEMS devices, integrated circuits, microprocessors, microcontrollers, angle measurement circuitry, RF circuits, and transceivers.

In some embodiments, high-aspect-ratio antennas can be deposited on dielectric substrates. The majority of current in accordance with certain embodiments may flow along the interface between the dielectric substrates and antennas. In such embodiments, a small surface roughness of the substrate/antenna interface enables a correspondingly low resistance in antennas. Methods to fabricate high-aspect-ratio antennas in accordance with several embodiments provide a smooth interface with small surface roughness without electroplating. Electroplating may reduce the resolution of structures formed on a substrate. In some embodiments, a thin electroplated layer disposed in one plating step can be used to provide a conductive coating on the conductor surfaces. The electrically conductive coating in accordance with some embodiments can be sufficiently thin as not to obscure the particle structure of the conductor. So that the conductor surface can have a bumpy, non-planar particle definition that conformally follows the contour of the underlying nanoparticle structure and exposes the nanoparticle structure of the conductor. Certain embodiments provide that thin electrically conductive layers can improve skin conduction of the electrical conductor while limiting loss in spatial resolution of the electrical conductors.

High-aspect ratio antenna structures in accordance with many embodiments enable longer and more-responsive antennas that improve signal response. In several embodiments, antenna windings can be formed more closely together. Some embodiments provide that the antenna windings can be formed closer than electroplated structures.

In many embodiments, high-aspect-ratio antenna structures provide a same inductance with decreased conductor line spacing for a given aspect ratio of an antenna in a smaller area with a smaller footprint. High-aspect-ratio antennas in accordance with several embodiments provide an increased inductance and signal sensitivity with decreased conductor line spacing and more turns for a given aspect ratio of an antenna compared to antennas with the same footprint but low aspect ratio. In a number of embodiments, the high aspect ratio of the conductors provides an increase in capacitance which is proportional to the aspect ratio.

Many embodiments provide high-aspect-ratio coil structures with high conductivity can be applied to the design and fabrication of high-Q, low-loss air-core inductors in high-frequency electronic circuit design. In several embodiments, the coil structures can be applied as inductors in areas including (but not limited to): switch mode power supplies, radio frequency (RF) band-pass, high-pass, and low-pass filters, low-loss transformers, inductive angle and position sensors, LC or RLC resonators. The printed inductors and/or coils in accordance with some embodiments can be integrated as discrete components, as part of a larger distributed element network, and/or microstrip containing multiple passive components. Some embodiments provide that the high accuracy of the printed inductors and/or coils can provide benefits including (but not limited to): more accurate tuning of the resonance frequency, smaller footprint, sub-quarter wavelength filtering, and higher power coupling efficiency.

Having described certain implementations of embodiments, it will now become apparent to one of skill in the art that other implementations incorporating the concepts of the disclosure may be used. Therefore, the disclosure should not be limited to certain implementations, but rather should be limited only by the spirit and scope of the following claims.

Throughout the description, where apparatus and systems are described as having, including, or comprising specific components, or where processes and methods are described as having, including, or comprising specific steps, it is contemplated that, additionally, there are apparatus, and systems of the disclosed technology that consist essentially of, or consist of, the recited components, and that there are processes and methods according to the disclosed technology that consist essentially of, or consist of, the recited processing steps.

It should be understood that the order of steps or order for performing certain action is immaterial so long as the disclosed technology remains operable. Moreover, two or more steps or actions in some circumstances can be conducted simultaneously. The invention has been described in detail with particular reference to certain embodiments thereof, but it will be understood that variations and modifications can be affected within the spirit and scope of the invention.

### Gas-Sensors

Gas sensors can be used to detect ambient gases and measure a gas concentration present in the atmosphere. Gases of interest can include toxic, explosive or environmental gases. Gas sensors may be used in a variety of applications, including industrial manufacturing, chemical-process control, nature conservation, personal-health monitoring, smart-city monitoring, indoor/outdoor air-quality control, and national defense.

Gas sensors can rely upon an attribute change of a gas-sensor element exposed to a target gas to which the corresponding gas-sensor element is sensitive. Gas sensors include a variety of different sensing architectures that transduce sensed gases into electrochemical, optical, acoustical, thermometric or gravimetric signals. Among these, electrically transduced gas sensors are one of the widely investigated and one of the common sensors. Electrical gas sensors may include two main components: a sensing material comprising the gas-sensor element and a transducer. The sensing material in the gas-sensor element may be exposed to the ambient atmosphere and, if a target gas is detected, undergoes a change in one or more of its physical properties, such as the conductivity, work function, or permittivity of the material. After the sensing material interacts with the target gas, the transducer converts the changed physical property into a change in the sensing material's electrical characteristics, such as capacitance (C), inductance (L), or resistance (R). A circuit then measures a magnitude, frequency (F), or phase ((p) variation in current (I) or voltage (V) corresponding to the change in the sensing material's electrical characteristics.

Electrically transduced gas sensors can be categorized into at least four different device architectures: resistive, capacitive, inductive, and field-effect-based gas sensor architectures. The electronic gas sensing materials are generally conductors or semiconductors and undergo electrical property changes when exposed to a target gas. Typical gas-sensing materials include metal-oxide semiconductors, conducting polymers, carbon nanotubes, and 2D materials. Most commercial gas sensors are based on metal-oxide semiconductor sensing layers, for example, NiO, SnO₂, TiO, WO₃, Fe₂O₃ and ZnO.

Metal-oxide gas sensors can be thick-film devices with a sensing layer thickness ranging from 1 μm to 100 μm or thin-film devices with a sensing layer thickness ranging from a few nm to 1 μm. The gas-sensing properties of the thin- and thick-film metal-oxide-based gas sensors of nominally the same material exhibit widely different responses to various gases at different temperature ranges.

Different techniques are currently used to deposit thin-film and thick-film layer metal-oxide sensing films. Deposition methods for thin-film deposition include vacuum deposition techniques such as physical vapor deposition, atomic layer deposition, molecular vapor deposition, thermal chemical vapor deposition, or flame spray pyrolysis. Thick-film deposition technologies include screen-printing, inkjet-printing, drop-casting, and electrohydrodynamic printing. Advanced and effective metal-oxide gas sensors may include nanostructured materials deposited as thick porous films on transducer electrodes.

Most commercial gas sensors may need a heater to sensitize the gas-sensor element to the gas. Since most gas sensors are intended for portable applications, power use by the gas sensor and the physical size of the gas sensor can be important performance attributes.

In previous work, Graf et al., discussed gas-sensitive metal-oxide materials including wide-bandgap semiconducting oxides such as tin oxide, gallium oxide, indium oxide, or zinc oxide. (See, e.g., M. Graf, et al., Journal of Nanoparticle Research, 2006, 8, 823-839; the disclosure of which is incorporated herein by reference in its entirety.) In general, gaseous electron donors or acceptors adsorb on the metal oxides and form surface states, which can exchange electrons with the semiconductor metal oxide. An acceptor molecule can extract electrons from the metal-oxide semiconductor and thus decrease its conductivity. The opposite holds true for an electron-donating molecule. A space-charge layer can thus be formed. By changing the surface concentration of donors/acceptors, the conductivity of the space-charge region can be modulated so that the conductivity of metal-oxide semiconductor materials changes in response to analyte gas-concentration changes. These chemically induced changes can then be transduced into electrical signals by means of simple electrode structures for making conductivity measurements.

Gas-sensor elements can comprise thin films formed by evaporation or comprise thick films formed by drop casting or screen printing a metal-oxide gas-sensor element or by depositing metal oxide nanoparticles in solution with an inkjet printer on a micro-heater. Gas-sensor elements can be constructed using micro-molding in capillaries (MIMIC) methods. (See, e.g., M. Heule, et al., Adv. Mater., 2001, 13, 23, 1790-1793; and M. Heule, et al., Sensors and Actuators B, 2003, 93, 1-3, 100-106; the disclosures of which are incorporated herein by references in their entirety.) However, such gas sensors have widely varying and inconsistent performance and can use more power than is desirable. Moreover, gas sensors that can simultaneously sense a variety of gases, for example in an electronic nose, may be useful.

Many embodiments provide gas sensors fabricated with micro-molding processes. Gas sensors in accordance with several embodiments have a smaller size and exhibit reduced power use. Several embodiments provide that the gas sensors are more consistent in performance. In some embodiments, the gas sensors include a wider variety of gas-sensor elements. The micro-molding fabrication processes in accordance with several embodiments are simple and repeatable manufacturing processes. Systems gas-sensors with high resolution gas-sensor elements that are fabricated with micro-molding processes in accordance with various embodiments of the invention are discussed further below.

### Micro-Molding Gas Sensors

Many embodiments provide structures and methods for making gas sensors. The gas sensors in accordance with several embodiments have lower power consumption, increased sensitivity, improved selectivity, increased consistency and controllability, reduced footprint, and simplified manufacturing processes. The footprint of a gas sensor is the area of the gas sensor over a substrate on which the gas sensor is disposed. At least one gas-sensor element can be constructed using micro-molding processes in accordance with some embodiments onto a surface. Many embodiments provide that the gas-sensor elements can comprise different materials and/or have different form factors. In some embodiments, the gas-sensor elements can be disposed on sensing electrodes over at least one micro-heater. The gas-sensor elements on sensing electrodes can be exclusively and directly over the at least one micro-heater. Several embodiments provide that the gas-sensor elements can be exposed to an ambient gas. In certain embodiments, the micro-heaters can heat the gas-sensor elements. A number of embodiments provide that the sensing electrodes can measure the electrical characteristics of the gas-sensor elements.

Many embodiments provide structures of gas-sensors made with micro-molding processes. In several embodiments, at least one element of the gas-sensors can be made with micro-molding processes. Micro-molded gas sensors can include at least one gas-sensor element. The gas-sensor elements can be nano-porous electrical conductors made of (but not limited to) fused nanoparticles. Fused nanoparticles in accordance with some embodiments can be sintered or welded nanoparticles. In some embodiments, micro-molded gas sensors include multiple gas-sensor elements. Gas-sensor elements can have an element length L, an element height H, and an element width W. In several embodiment, gas-sensor element height H can be greater than element width W. In certain embodiments, electrodes can be electrically connected to gas-sensor elements. Some embodiments refer the electrodes as gas-sensor electrodes. In a number of embodiments, additional current- and/or voltage-injection force electrodes can be incorporated. Force electrodes in accordance with several embodiments can connect to gas-sensor elements to provide a 4-point probe measurement configuration. Such embodiments can improve the long-term stability of the gas-sensors by decreasing or eliminating the influence of the contact resistance between the sensing elements on the measurement. Electrical characteristics of gas-sensor elements in accordance with many embodiments can change in response to an ambient gas in contact with the nano-porous electrical conductors. The electrical characteristics can include (but are not limited to) resistivity, capacitance, inductance, phase, and any combinations thereof. In several embodiments, gas-sensors can include sensor controllers electrically connected to electrodes. In some embodiments, sensor controllers can be operable to provide electrical current to, and measure the resistivity of, gas-sensor elements through the electrodes and/or other electrical connections to gas-sensor elements.

In some embodiments, gas sensors can include multiple gas-sensor elements that can be substantially identical (for example within manufacturing tolerances). Multiple, substantially identical gas-sensor elements in accordance with several embodiments can provide redundant measurements that can be combined to reduce variability and improve consistency and accuracy in gas sensor measurements. Certain embodiments provide that each gas-sensor element can be connected by a separate first electrode and a separate second electrode to a sensor controller. A number of embodiments provide that gas-sensor elements can be electrically connected to a common first electrode and/or a common second electrode.

Many embodiments provide that gas sensors can be disposed on at least one micro-heater on a substrate. In several embodiments, the micro-heaters can be electrical micro-heaters. In some embodiments, electrical micro-heaters can include at least one micro-heater electrode including (but not limited to) a resistive electrical conductor, or resistive wire. Certain embodiments provide at least one electrically insulating layer including (but not limited to) a dielectric layer, or a SiO₂ layer, can be disposed on the micro-heaters and gas-sensor elements can be disposed on the insulating layers. Insulating layers in accordance with some embodiments can electrically insulate and protect gas-sensor-elements from micro-heater electrodes. In several embodiments, micro-heaters can extend beyond gas-sensor elements in one or two orthogonal directions, so that gas-sensor elements can be surrounded by micro-heaters in a horizontal direction parallel to a surface on or over the substrate. A number of embodiments provide that the surface can be a surface of the substrate on which gas-sensor elements are disposed, or a surface of any layer disposed on the substrate on which gas-sensor elements are disposed. By uniformly heating the part of the gas-sensor elements by microheaters, which is located between the sensing electrodes and/or force electrodes, the temperature of gas-sensor elements can be more consistent and better controlled and can provide more reliable and consistent electrical characteristic measurements in accordance with some embodiments.

In many embodiments, micro-heaters can provide heat to gas-sensor elements to control the temperature of gas-sensor elements. Heat can decrease the resistivity of the gas sensor elements and enhance the interaction of target gas molecules with the sensing material and can therefore increase the sensitivity of the sensor elements to the target gas. In several embodiments, gas-sensor elements can operate more effectively at elevated temperatures including (but not limited to) temperatures greater than ambient and/or room temperature, such as between about 150° C. and about 350° C. Micro-heaters in accordance with embodiments can be electrically connected to and controlled by sensor controls.

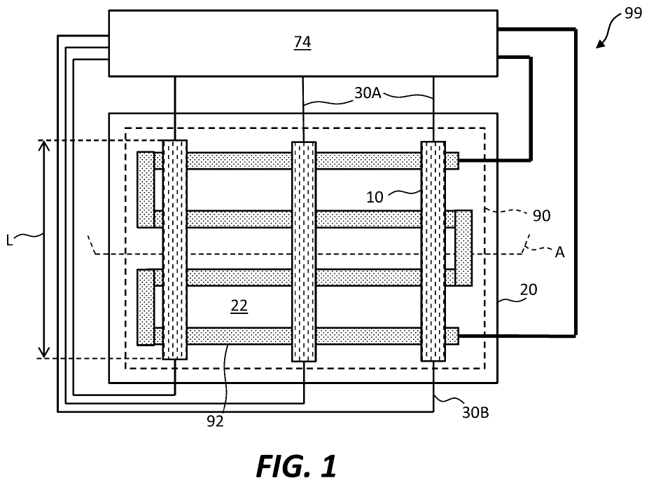

A plan view of a gas-sensor in accordance with an embodiment of the invention is illustrated in FIG. 1. A micro-molded gas sensor 99 includes at least one gas-sensor element 10. The gas-sensor element 10 can include a nano-porous electrical conductor comprising fused nanoparticles. Gas-sensor element 10 can have an element length L, an element height H, and an element width W. At least a first electrode 30A can be electrically connected to a gas-sensor element 10, for example at a first end of a gas-sensor element 10. At least a second electrode 30B can be electrically connected to the gas-sensor element 10, for example at a second end of the gas-sensor element 10 opposite from the first end. First electrodes 30A and second electrodes 30B are collectively referred to as gas-sensor electrodes 30. An electrical characteristic of gas-sensor element 10 changes in response to an ambient gas in contact with the nano-porous electrical conductor. In some embodiments the gas sensor 99 can comprise a sensor controller 74 electrically connected to the first electrodes 30A and electrically connected to the second electrodes 30B. The sensor controller 74 can be operable to provide electrical current to, and measure the resistivity of, gas-sensor element 10, for example through first electrodes 30A and second electrodes 30B or other electrical connections to gas-sensor element 10.

In FIG. 1, The gas sensor 99 can comprise a substrate 20, and a micro-heater 90 disposed on the substrate 20. The electrical micro-heater 90 can include at least one micro-heater electrode 92. such as a resistive electrical conductor or resistive wire) disposed on substrate 20. Micro-heater 90 can be electrically connected to and controlled by sensor control 74.

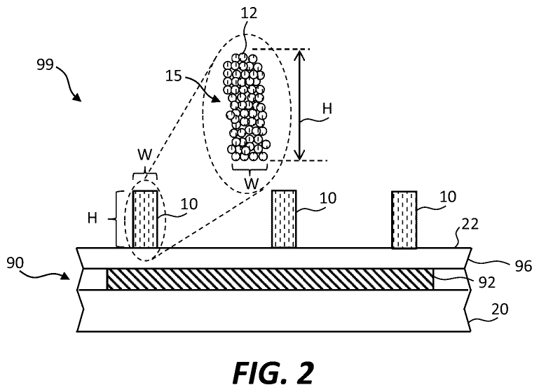

A cross section view of a gas-sensor taken along cross section line A of FIG. 1 in accordance with an embodiment of the invention is illustrated in FIG. 2. A micro-molded gas sensor 99 includes at least one gas-sensor element 10. The gas-sensor element 10 can include a nano-porous electrical conductor comprising fused nanoparticles 12. Fused nano-particles 12 can be sintered or welded nano-particles 12. Gas-sensor element 10 can have an element length L, an element height H, and an element width W. Element height H can be greater than element width W.

In FIG. 2, gas sensor 99 can comprise a substrate 20. A micro-heater 90 comprising a micro-heater electrode 92 such as a resistive electrical conductor or resistive wire can be disposed on the substrate 20. An electrically insulating layer 96 (e.g., a dielectric such as SiO₂) can be disposed on the micro-heater 90 and gas-sensor elements 10 can be disposed on insulating layer 96. Insulating layer 96 electrically insulates and protects gas-sensor-elements 10 from micro-heater electrodes 92. Micro-heater 90 can extend beyond gas-sensor element 10 so that gas-sensor elements 10 are surrounded by micro-heater 90 in a horizontal direction parallel to a surface 22 on or over substrate 20. By surrounding gas-sensor elements 10 by micro-heaters 90, the temperature of gas-sensor elements 10 can be more consistent and better controlled and can provide more reliable and consistent electrical characteristic measurements. Although FIG. 1 and FIG. 2 illustrate specific gas-sensor structural schemes and gas-sensor element compositions, any configuration and design can be utilized as appropriate depending on the specific requirements of the given application.

Many embodiments provide that gas-sensors made with micro-molding processes can include gas-sensor elements that are different from each other. Several embodiments provide that multiple different gas-sensor elements in a gas sensor can provide measurements of different gases and/or gas concentrations in a single gas sensor. Some embodiments implement gas sensors in electronic noses. Multiple different gas-sensor elements in accordance with many embodiments can comprise electrical conductors made of different nanoparticles. Different nanoparticle compositions of multiple different gas-sensor elements in accordance with certain embodiments can be sensitive to different gases and/or gas concentrations. In several embodiments, the selectivity of gas-sensor elements can be characterized by the ratio of the output signal change in the presence of the target gas and the output signal change when a different gas is present.

In many embodiments, multiple different gas-sensor elements of gas sensors can be made of different nanoparticles. The different nanoparticles in accordance with several embodiments can include (but are not limited to) different nanoparticle materials, different nanoparticle doping, different nanoparticle sizes, and any combinations thereof. The nano-porous electrical conductors of different gas-sensor elements in accordance with some embodiments can have different nano-porosities including (but not limited to) nanopore sizes, and quantities of nano-pores in the nano-porous electrical conductors.

In some embodiments, gas-sensor elements of a gas-sensor can have the same/or different form factors than other gas-sensor elements of the same gas-sensor. Examples of form factors include (but not limited to) length of a gas-sensor element, height of a gas-sensor element, width of a gas-sensor element, and an element shape.

Many embodiments provide that gas-sensor elements can have element height H greater than element width W. In some embodiments, the ratio between the element height H and the element width W can be up to and greater than 2. In several embodiments, the ratio between the element height H and the element width W can be up to and greater than 4, can be up to and greater than 8, can be up to and greater than 16. In certain embodiments, the ratio between the element height H and the element width W can be less than 0.5, can be less than 0.25. Gas-sensor elements having an increased element height H with respect to element width W (e.g., an increased aspect ratio) in accordance with many embodiments can have an increased gas-sensor-element surface area. Several embodiments provide that gas-sensors elements with increased surface area can be disposed closer together in a reduced area over a substrate, reducing the footprint of gas sensor. In some embodiments, the ratio between the element width and the spacing between the elements can be less than 4. In some embodiments, electrical characteristics response of gas-sensor elements can be dependent, at least in part, on gas-sensor-element-surface area including (but not limited to) the surface area of the nano-porous electrical conductor. In a number of embodiments, nano-porous electrical conductors can increase an interfacial area between the sensing material and a gas such that the response of gas-sensor elements to a corresponding gas can be increased. Several embodiments provide that gas sensors comprising high-aspect ratio nano-porous gas-sensor elements have increased sensitivity and a reduced footprint.

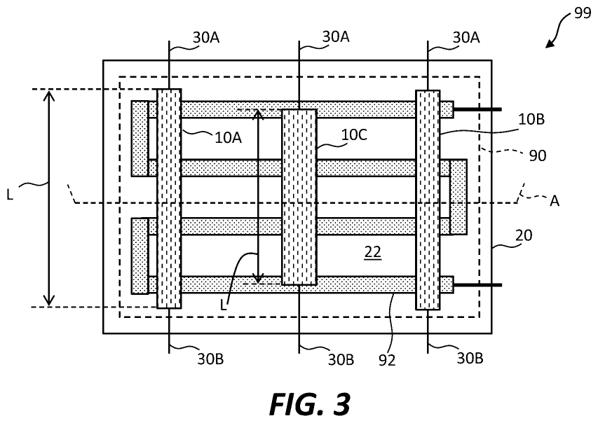

A plan view of a gas-sensor with different gas-sensor elements in accordance with an embodiment of the invention is illustrated in FIG. 3. The gas sensor 99 comprises multiple gas-sensor elements 10A, 10B, 10C that are different from each other. Multiple gas-sensor elements 10A-C in gas sensor 99 can provide measurements of different gases and/or gas concentrations in a single gas sensor 99. For example, a first gas-sensor element 10A can comprise nanoparticles that are different from the nanoparticles in and a second gas-sensor element 10B. The nanoparticles can be sensitive to different gases and/or gas concentrations.

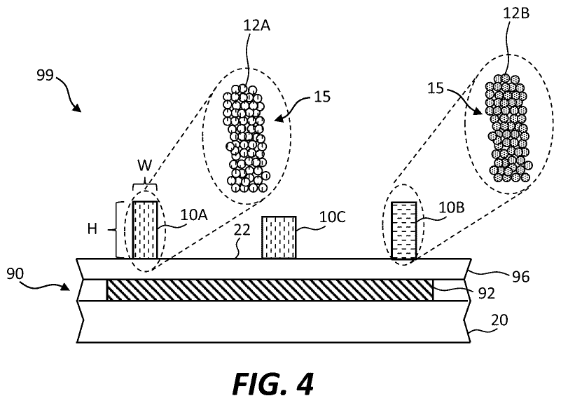

A cross section view of a gas-sensor with different gas-sensor elements taken along cross section line A of FIG. 3 in accordance with an embodiment is illustrated in FIG. 4. The gas sensor 99 comprises multiple gas-sensor elements 10A, 10B, 10C that are different from each other. Multiple gas-sensor elements 10A-C can provide measurements of different gases and/or gas concentrations in a single gas sensor 99. A first gas-sensor element 10A can comprise first nanoparticles 12A and a second gas-sensor element 10B can comprise second nanoparticles 12B that are different from first nanoparticles 12A and are sensitive to different gases or gas concentrations. First and second nanoparticles 12A, 12B are collectively referred to as nanoparticles 12. Third gas-sensor element 10C can also comprise different nanoparticles 12 (not shown). The gas-sensor element 10C can have a different form factor than the first gas-sensor element Different form factors can be: a different length L (length between first electrode 30A and second electrode 30B electrical connections to gas-sensor element 10 as shown in FIG. 3), a different cross section (e.g., a different element height H, a different element width W, or a different element shape).

In FIG. 4, gas-sensor elements 10A and 10B having an increased element height H with respect to element width W (e.g., an increased aspect ratio) can have an increased gas-sensor-element surface 15 area and can be disposed closer together in a reduced area over a substrate 20 on which gas-sensor element 10 is disposed, reducing the footprint of gas sensor 99. An electrical characteristic response of gas-sensor element can be dependent, at least in part, on gas-sensor-element-surface 15 area, for example the surface area of the nano-porous electrical conductor. The nano-porous electrical conductor of gas-sensor elements can improve the interfacial area between the sensing material and the gas and the response of gas-sensor elements to a corresponding gas is increased. Although FIG. 3 and FIG. 4 illustrate specific gas-sensor structural schemes and multiple different gas-sensor element compositions, any configuration and design can be utilized as appropriate depending on the specific requirements of the given application.

In many embodiments, micro-heaters of gas-sensors can include individually controllable micro-heater segments. The individually controllable micro-heater segments in accordance with several embodiments can be individually controllable by including (but not limited to) sensor controllers to provide a different temperature in each micro-heater segment simultaneously. Some embodiments provide that each micro-heater segment can be associated with and/or in thermal contact with a different gas-sensor element. In such embodiments, the multiple micro-heater segments can simultaneously heat corresponding gas-sensor elements to different temperatures. Gas-sensor elements heated to different temperatures in accordance with many embodiments can be applied to detect different gases and/or concentrations of gases, through individual and separate micro-heater electrodes. Several embodiments provide that substrates and/or insulating layers can have a relatively high thermal resistance so as to enable different temperatures in different micro-heater segments. By providing differently controllable and different gas-sensor elements, gas sensors can measure different gases and/or different gas concentrations at the same time and can comprise an electronic nose.

By decreasing the footprint of gas sensor elements, a total area of the microheater can be decreased in accordance with many embodiments without compromising the temperature uniformity of gas-sensor elements. Microheater power draw may increase with area. The decrease of the total area of the microheater can result in a decrease in gas sensor power consumption, facilitating the use of gas sensors in accordance with several embodiments in battery-powered electronics.

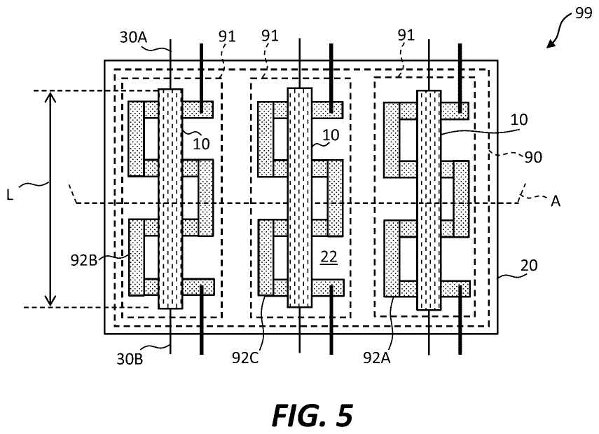

A plan view of individually controllable micro-heater segment in a gas-sensor in accordance with an embodiment of the invention is illustrated in FIG. 5. Micro-heater 90 comprises individually controllable micro-heater segments 91 that are individually controllable (e.g., by sensor controller 74 as shown in FIG. 1) to provide a different temperature in each micro-heater segment 91 simultaneously. Each micro-heater segment 91 can be associated with or in thermal contact with a different gas-sensor element 10 and the multiple micro-heater segments 91 can simultaneously heat corresponding gas-sensor elements 10 to different temperatures, for example to detect different gases or concentrations of gas, through individual and separate micro-heater electrodes 92 (e.g., first micro-heater electrode 92A, second micro-heater electrode 92B, third micro-heater electrode 92C, collectively micro-heater electrodes 92). Substrate 20, insulating layer 96, or both, can have a relatively high thermal resistance so as to enable different temperatures in different micro-heater segments 91. By providing differently controllable and different gas-sensor elements 10, gas sensor 99 can measure different gases or different gas concentrations at the same time and can comprise an electronic nose. Decreasing the footprint of gas sensor element 10 can decrease a total area of the microheater 90 without compromising the temperature uniformity of gas-sensor element 10. As the microheater 90 power draw increases with area, this results in a decrease in gas sensor 99 power consumption, facilitating the use of such gas sensors 99 in battery-powered electronics. Although FIG. 5 illustrates specific micro-heater incorporation in gas-sensor schemes, any configuration and design can be utilized as appropriate depending on the specific requirements of the given application.

In many embodiments, gas sensors can be micro-sensors having elements that have sizes in the range from nanometers, microns, to tens of microns. In some embodiments, gas-sensor elements can have an element width W ranging from about 1 micron to about 50 microns. In certain embodiments, gas-sensor elements can have an element width W ranging from about 5 microns to about 20 microns. In a number of embodiments, the width W of a gas-sensor element can be about 10 microns. Several embodiments provide that gas-sensor elements can have an element height H ranging from about 100 nanometers to about 20 microns. In some embodiments, gas-sensor elements can have a height H ranging from about 1 micron to about 10 microns. In certain embodiments, the height H of gas-sensor elements can be about 5 microns. In many embodiments, gas-sensor elements can be separated over the substrate by a distance that ranges from about 1 micron to about 50 microns. Several embodiments provide that gas-sensor elements can be separated over the substrate by a distance that ranges from about 5 microns to about 20 microns. In some embodiments, gas-sensor elements can be separated over the substrate by a distance about 15 microns. Many embodiments provide that gas-sensor electrodes can have a thickness ranging from about 10 nm to about 5 microns. In several embodiments, gas-sensor electrodes can have a thickness of about 100 nm. In some embodiments, each gas-sensor element can have a height H in the range from about 1 micron to about 20 microns, a width in the range from about 1 micron to about 50 microns.

The sizes and separation of gas-sensor elements and gas-sensor electrodes in accordance with several embodiments may not be constructed using ink-jet, drop-casting, and/or screen-printing techniques. In addition, thin-film structures may have reduced surface area and therefore reduced sensitivity. Many embodiments enable gas sensors with increased sensitivity and reduced footprint. Several embodiments provide that gas-sensor elements can be constructed more repeatably and with better control of the amount and structure of the sensing material including (but not limited to) fused nanoparticles between electrodes so that electrical characteristic measurements can be more consistent and repeatable. Improved fabrication fidelity and reproducibility in accordance with several embodiments can lead to better control of the surface properties of gas-sensor elements, thereby decreasing the variability of electrical response of manufactured gas sensors and mitigating calibration needs of gas sensors after manufacture.

Many embodiments provide that gas-sensor elements of gas-sensors can have geometric shapes including (but not limited to) linear and straight line, curved, or spiral. A single gas-sensor element with a single first electrode and a single second electrode in accordance with several embodiments can have multiple portions that each correspond to a different nano-porous electrical conductor. In some embodiments, multiple gas-sensor elements can be interdigitated and/or different gas-sensor elements can have different nano-porous electrical conductors that are interdigitated. Gas-sensor elements can have different shapes and/or cross sections including (but not limited to) square, rectangular, cubic, circular, or cylindrical. In several embodiments, gas-sensor elements having a linear form factor including (but not limited to) a high-aspect form factor and an element length L much greater than an element width W or element height H, can have a greater gas-sensor-element surface area in a reduced area of surface. Gas-sensor elements in accordance with certain embodiments have a greater gas-sensor-element surface area in contrast with gas-sensor materials provided using drop-casting, screen printing, or ink-jet printing. Gas-sensor elements with greater gas-sensor-element surface area in accordance with a number of embodiments can increase the sensitivity of the gas-sensor elements and reduce the size of gas sensors, particularly for gas sensors comprising multiple gas-sensor elements in an electronic nose.

Many embodiments provide that nanoparticles of gas-sensor elements can have diameters ranging from about 1 nm to about 1 micron. In some embodiments, the nanoparticles can have diameters ranging from about 10 nm to about 500 nm. In several embodiments, the nanoparticles can have diameters of about 100 nm or less than about 100 nm. In certain embodiments, the nanoparticles have diameters ranging from about 1 nm to about 5 microns.

In many embodiments, assemblies of nanoparticles in the nano-porous electrical conductors of gas-sensor elements may not be identical and may have a distribution of sizes. Distribution of nanoparticle sizes in accordance with several embodiments can include (but are not limited to) a distribution of diameters, centered about a nominal diameter. Thus, nanoparticles referred to as having a diameter of about 100 nm in accordance with embodiments can be actually a collection or assembly of nanoparticles having a distribution of diameters substantially with an average of about 100 nm (e.g., within manufacturing tolerances). Several embodiments provide that gas-sensor elements can have a surface roughness of less than about 1 micron RMS. In certain embodiments, gas-sensor elements can have a surface roughness of less than about 150 nm RMS, less than about 100 nm RMS, and/or less than about 50 nm RMS.

Many embodiments provide that nanoparticles of gas sensors can include (but are not limited to) metal nanoparticles, metal-oxide nanoparticles, or doped metal-oxide nanoparticles. Metal-oxide nanoparticles in accordance with certain embodiments can include (but are not limited to) SnO₂, TiO₂, ITO, CdSe, WO₃, ZnO, In₂O₃, Cd:ZnO, CrO₃, V₂O₅, and any combinations thereof. In some embodiments, metal-oxide nanoparticles can be doped with Al, Pt, Pd, Au, Ag, Ti, Cu, Fe, Sb, Mo, Ce, Mn, Rh₂O₃, or carbon nanotubes (CNTs) to improve the selectivity of the sensor. Such materials can be effectively used to detect gases of interest in various applications. In several embodiments, assemblies of nanoparticles can comprise materials including (but not limited to) non-conductive materials, semi-conducting, and/or dielectric materials. These materials in accordance with embodiments can be sensitive to gases and affect the response of conductive materials in the nano-porous electrical conductor, and/or can be useful for constructing the nano-porous electrical conductor. In a number of embodiments, nanoparticle ink can be provided as a suspension in liquid solvent including (but not limited to) aqueous dispersants, and/or organic solvents. Examples of organic solvents include (but are not limited to) isopropanol, ethanol, toluene, ethylene glycol, propylene glycol, diethylene glycol, triethylene glycol, diethylene glycol monomethyl ether, or triethylene glycol monomethyl ether. Nanoparticles in accordance with several embodiments can have viscosities in a range from about 0.3 centipoise to about 300 centipoises. In some embodiments, nanoparticles comprise different nanoparticles made of different conductive, semi-conducting, or non-conductive materials and can be distributed isotropically or anisotropically in gas-sensor elements.

Many embodiments provide that substrates of gas-sensors can include (but are not limited to) glass, polymers, semiconductors, ceramics, quartz, metals, paper, and/or sapphire. Examples of polymers comprising the substrates can include (but are not limited to) Kapton (polyimide), PET, PMMA, Teflon (PTFE), and ETFE. Examples of semiconductors can include (but are not limited to) Si, SiO₂, Si₃N₄, SiC, GaAs, GaInP, In P, and any combinations of these materials. In several embodiments, the substrates for gas-sensors can be a printed-circuit board (PCB) substrate including (but not limited to) FR2, FR4, or liquid-crystal polymer (LCP) materials. Some embodiments provide that the substrates can be rigid, flexible, and/or substantially planar. In a number of embodiments, the substrates can be found in the display, integrated circuit, electronics assembly, or circuit board industries. In some embodiments, the substrates may contain CMOS and/or MEMS devices, integrated circuits, microprocessors, microcontrollers, angle measurement circuitry, RF circuits, and transceivers.

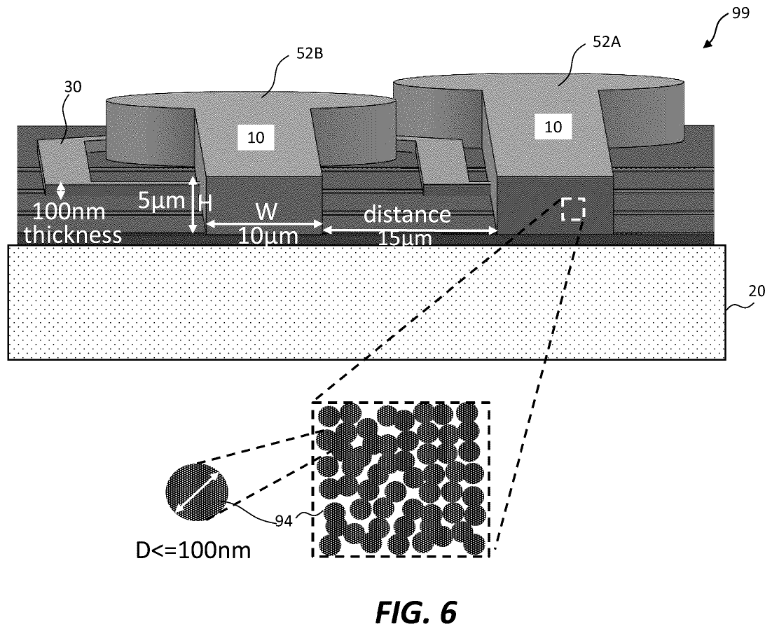

A gas sensor having gas-sensor elements with various sizes in accordance with an embodiment of the invention is illustrated in FIG. 6. Gas sensor 99 can be a micro-sensor having elements that have sizes in the nanometer, micron, or tens of microns range. For example, gas-sensor elements 10 can have an element width W ranging from about 1 micron to about 50 microns or ranging from about 5 microns to about 20 microns. In FIG. 6, the width W of one gas sensor element 10 is about 10 microns. Gas-sensor elements 10 can have an element height H ranging from about 100 nanometers to about 20 microns or ranging from about 1 micron to about 10 microns. In FIG. 6, the height H of one gas-sensor element 10 is about 5 microns. Gas-sensor elements 10 can be separated over the substrate 20 by a distance that ranges from about 1 micron to about 50 microns or ranges from about 5 microns to about 20 microns. In FIG. 6, the distance separation between gas-sensor elements is about 15 microns. Gas-sensor electrodes 30 can have a thickness ranging from about 10 nm to about 5 microns. In FIG. 6, the thickness of gas-sensor electrode is about 100 nm. Gas-sensor elements 10 can be made with nanoparticles 94. Nanoparticles 94 can have a range of diameters D, for example ranging from about 1 nm to about 1 micron, or ranging from about 10 nm to about 500 nm. In FIG. 6, nanoparticles 94 have diameters of about 100 nm or less than about 100 nm, for example about 1 nm to about 5 microns. Although FIG. 6 illustrates specific gas-sensor structural dimensions and gas-sensor element compositions, any configuration and design can be utilized as appropriate depending on the specific requirements of the given application.

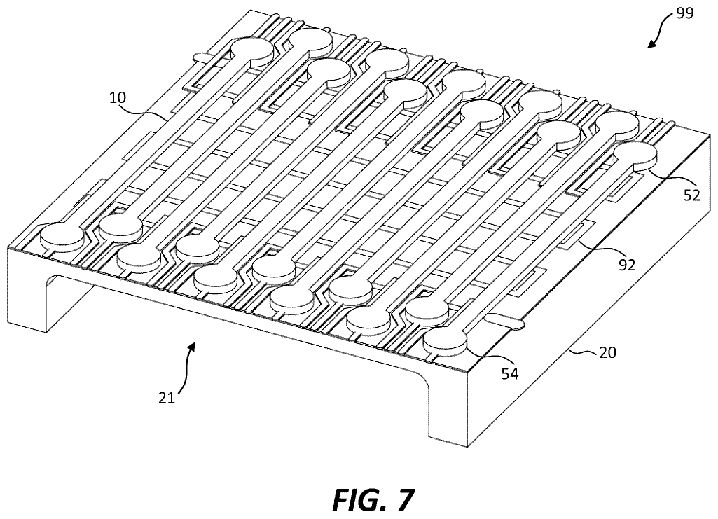

In many embodiments, gas-sensors can have a substrate with sections of different thickness. Substrate with a thinner section in accordance with some embodiments can provide faster temperature changes and better heat control and temperature distribution to gas-sensor elements in response to micro-heaters. A gas-sensor with a substrate with sections of different thickness in accordance with an embodiment is illustrated in FIG. 7. The substrate 20 can have a thinner substrate 21 at a center of the substrate 20 than at an edge of substrate 20. Such a thinned substrate 21 can provide, for example, faster temperature changes and better heat control and temperature distribution to gas-sensor elements 10 in response to micro-heater 90, by reducing the heat loss through the substrate 20. In some embodiments thinner substrate 21 can comprise a SiO₂ or Si₃N₄ membrane, with a thickness between about 10 nm and about 1 micron, which can be suspended over an aperture in substrate 20. In some embodiments, this membrane may contain openings to further reduce heat losses.

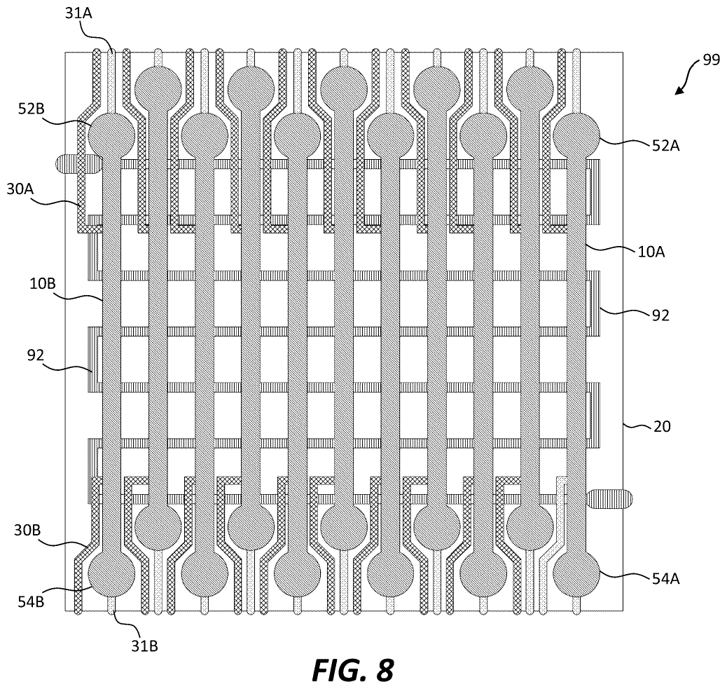

Many embodiments provide that gas sensors can be constructed using micro-molding machines (discussed further below). A plan view of a gas sensor disposed on or over a substrate in accordance with an embodiment of the invention is illustrated in FIG. 8. In FIG. 8, the gas-sensor 99 comprises multiple gas-sensor elements 10 (e.g., first and second gas-sensor elements 10A, 10B). Each gas-sensor element has first and second electrodes 30A, 30B disposed over a micro-heater electrode 92 on substrate 20 (insulating layer 96 is not shown). First and second electrodes 30A, 30B provide conductivity sensing of the corresponding gas-sensor elements 10 and conduct current through the nano-porous electrical conductor of each gas-sensor element 10.

As shown in FIG. 8, additional current- or voltage-injection force electrodes 31A, 31B can be incorporated. Force electrodes 31A 31B can be connected to gas-sensor elements 10 to provide a 4-point probe measurement configuration. This may improve the long-term stability of the device by decreasing or eliminating the influence of the contact resistance between the sensing element (e.g., first and second electrodes 30A, 30B) on the measurement. Although FIG. 7 and FIG. 8 illustrate specific gas-sensor structures and compositions, any configuration and design can be utilized as appropriate depending on the specific requirements of the given application.

Systems of micro-molding machines that can be utilized in the micro-molding processes in accordance with various embodiments of the invention are discussed further below.

### Micro-Molding Machines

Many embodiments provide micro-molding machines that can be used in the micro-molding processes. Micro-molding machines in accordance with several embodiments can fabricate high resolution electrical conductors with high-aspect-ratio. The electrical conductors can be integrated in electrical devices including (but not limited to) gas sensors, inductors, antennas. Many embodiments provide that micro-molding machines have various features embedded in at least one surface of the micro-molding machines. In some embodiments, the micro-molding machines can act as stamps to imprint the embedded features onto a substrate. In several embodiments, the micro-molding machines have at least one ink supply to supply the ink during micro-molding processes.

Many embodiments provide that micro-molding machines include at least a stamp. The stamp can have a first channel disposed on a surface of the stamp and a second channel disposed on the surface of the stamp in accordance with some embodiments. In some embodiments, a first inlet port can be connected to the first channel, and a second inlet port separate from the first inlet port can be connected to the second channel. Certain embodiments provide a first ink including (but not limited to) nanoparticle ink supply for supplying a first ink to the first inlet port, and a second ink including (but not limited to) nanoparticle ink supply separate from the first ink supply for supplying a second ink to the second inlet port. In some embodiments, micro-molding machines include a pump and/or dispenser for pumping and/or dispensing the first ink through the first inlet port and the first channel, and for pumping and/or dispensing the second ink through the second inlet port and the second channel. Micro-molding machines in accordance with many embodiments can have a contact mechanism for contacting the surface of the stamp to a substrate. In some embodiments, channels within the stamp can be positioned with respect to features on the substrate to ensure that features are disposed at specific locations on the substrate, within a specified position tolerance including (but not limited to) 1 micron, or 10 microns. In certain embodiments, channels within the stamp can be positioned with respect to features on the substrate optically using reference markers on the stamp and substrate. In a number of embodiments, channels within the stamp can be positioned with respect to features on the substrate by mechanical contact.

In several embodiments, the first ink can be an ink comprising nanoparticles and the second ink can be an ink comprising nanoparticles, and the nanoparticles compositions in the first ink can be the same or different from the nanoparticle compositions in the second ink. Some embodiments provide that the first channel can have a first form factor and the second channel can have a second form factor that is the same or different from the first form factor. In many embodiments, micro-molding machines can include an outlet port connected to the first channel or the second channel. The pump and/or dispenser in accordance with several embodiments can provide a negative air pressure or vacuum that is less than an atmospheric pressure to the outlet port.

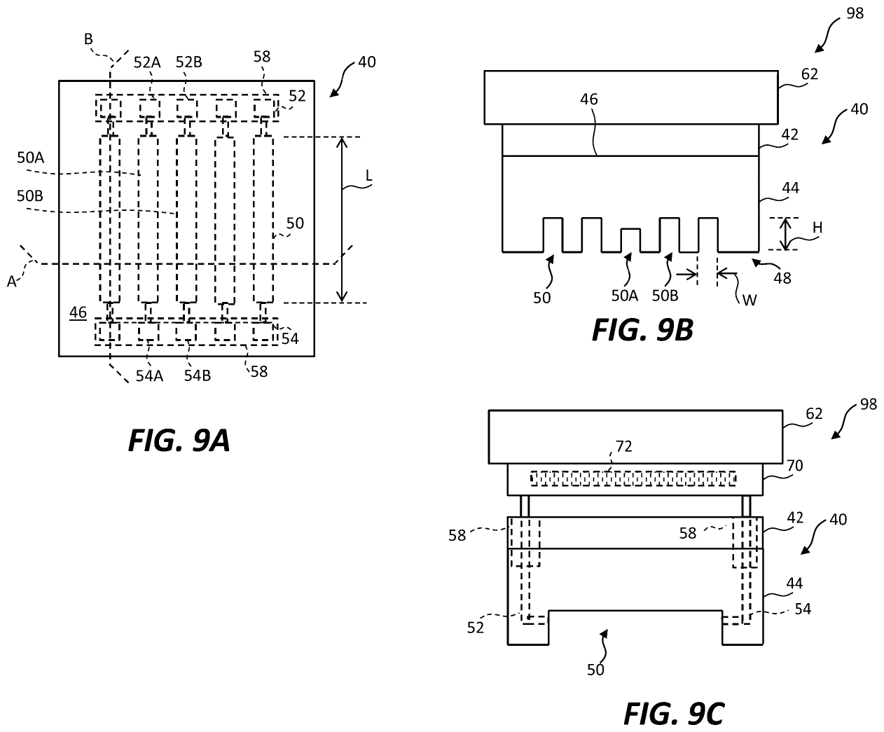

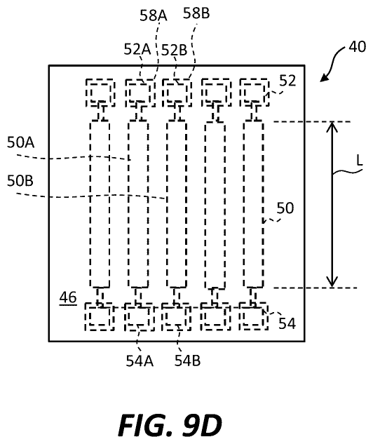

Several embodiments provide gas-sensors can be constructed using micro-mold machines. A plan view of a micro-mold machine in accordance with an embodiment of the invention is illustrated in FIG. 9A. A cross section view of the micro-mold machine taken across the cross-section line A of FIG. 9A is illustrated in FIG. 9B. A cross section view of the micro-mold stamp taken across the cross-section line B of FIG. 9A is illustrated in FIG. 9C. A plan view of a micro-mold machine with separate ink reservoirs in accordance with an embodiment of the invention is illustrated in FIG. 9D.

Micro-mold machine 98 can include a micro-mold stamp 40 that comprises a mold layer 44 having a support side 46 and a channel side 48. A support layer 42 is disposed in contact with support side 46. Support layer 42 can be more rigid than mold layer 44 to provide dimensional stability to mold layer 44 and enable improved resolution for structures formed by micro-mold stamp 40. Mold layer 44 can comprise at least one channel 50 (e.g., a micro-channel or multiple channels 50, as shown) disposed on the channel side 48 in mold layer 44. An inlet port 52 is connected to the channel 50, and an outlet port 54 is connected to the channel 50. Channel 50 has a height in a direction into mold layer 44 away from channel side 48 toward support side 46 (corresponding to gas-sensor element height H). The channel height can be greater than a width of the channel on the channel side 48 (corresponding to gas-sensor element width W). In some embodiments, inlet and outlet ports 52, and/or 54 can extend to channel side 48 surface of the mold layer 44. Inlet ports 52 provide a path for nanoparticle inks 56 to enter channels 50 and outlet ports 54 provide a path for nanoparticle inks to be drawn into or out of channels 50. Mold layer 44 can comprise an elastomeric material including (but not limited to) polydimethylsiloxane polyurethane, room-temperature vulcanizing silicone rubber, or photocurable rubbers cast and cured on a defined master, for example a master structure micromachined into a silicon wafer, or a polymer structure fabricated onto a substrate such as a silicon wafer, for example by means of photolithography. Support layer 42 can comprise a more rigid material than mold layer 44, for example glass, silicon, polymethylmethacrylate, polycarbonate, or quartz and can be thinner than mold layer 44. In some embodiments, mold layer 44 can be reinforced by incorporation of nanoparticles into the elastomeric material, or by the inclusion of a fiber mesh composed of including (but not limited to) glass, steel, carbon, or nylon. Support layer 42 can comprise a more rigid material including (but not limited to) glass, than mold layer 44, and can be thinner than mold layer 44.

In FIGS. 9A-9D, a micro-molding machine 98 comprises a micro-mold stamp 40 having a first channel 50A disposed on a surface of micro-mold stamp 40 and a second channel 50B disposed on the surface of micro-mold stamp 40. (First channel 50A and second channel 50B are collectively channels 50.) In some embodiments, channels 50 have a common, substantially identical form factor (as shown in FIGS. 9A, 9D). In some embodiments, first channel 50A has a first form factor and second channel 50B has a second form factor different from the first form factor (as shown in FIG. 9B). A first inlet port 52A is connected to first channel 50A and a second inlet port 52B is connected to second channel 50B.

In some embodiments, each channel 50 has a separate and individual inlet port 52 (e.g., first inlet port 52A and second inlet port 52B) and a separate and individual outlet port 54 (e.g., first outlet port 54A and second outlet port 54B). In some embodiments, the individual inlet and outlet ports 52, 54 to each channel 50 can be positioned in the stamp such that the minimum spacing between any pair of ports is greater than a predefined distance ranging from about 100 microns to about 1 mm. In some embodiments, multiple channels 50 share an inlet port 52, an outlet port 54, or both. A common inlet port 52 provides structural simplicity and manufacturability when channels 50 connected to the common inlet port 52 share a common nanoparticle 12 material. A common outlet port 54 provides structural simplicity and manufacturability for drawing nanoparticle 12 material from channels 50 connected to the common outlet port 54. In some embodiments the channels connected to common inlet port 52 and outlet port 54 are used to deposit nanoparticle ink for gas sensor elements which are part of multiple, separate gas-sensors which are constructed on a common substrate 20 using a single micro-mold stamp 40.

In FIG. 9A, first inlet port 52A is fed from a nanoparticle ink (not shown) supply (ink reservoir 58) and second inlet port 52B is fed from the same nanoparticle ink supply (ink reservoir 58) so that the same nanoparticles can be supplied to both first inlet port 52A and second inlet port 52B and supplied to both first channel 50A and second channel 50B.

In FIG. 9D, first inlet port 52A is fed from a nanoparticle ink supply (first ink reservoir 58A) and second inlet port 52B is fed from a different nanoparticle ink supply (second ink reservoir 58B) so that the different nanoparticles (e.g., first nano-particles 12A and second nano-particles 12B, not shown) can be supplied separately to first inlet port 52A and second inlet port 52B and separately to first channel 50A and second channel 50B. (First ink reservoir 58A and second ink reservoir 58B are collectively ink reservoirs 58. Inlet ports 52 and outlet ports 54 can comprise an ink reservoir 58.)

In FIG. 9C, a pump 70 or dispenser pumps and/or dispenses the first nanoparticle ink through first inlet port 52A and first channel 50A and the second nanoparticle ink through the second inlet port 52B and second channel 50B. In some embodiments, first and second nanoparticle inks can be the same nanoparticle ink or different nanoparticle inks. Pump or dispenser 70 can provide pressure to infill nanoparticle inks in channels 50 at a greater speed and with reduced costs than applying capillary action alone. A contact mechanism (e.g., an opto-mechatronic motion-control platform 62, shown in FIG. 9B, employing mechanical position micro-controllers and position sensors, e.g., optical sensors) can contact the surface (e.g., channel side 48) of micro-mold stamp 40 onto surface 22 (e.g., substrate 20 or a layer on substrate 20 such as insulating layer). Thus, a micro-molding machine 98 can provide different nanoparticle inks to substantially identical channels 50, provide substantially identical nanoparticle inks to different channels 50 (e.g., channels 50 having a different form factor), or provides different nanoparticle inks to different channels 50.

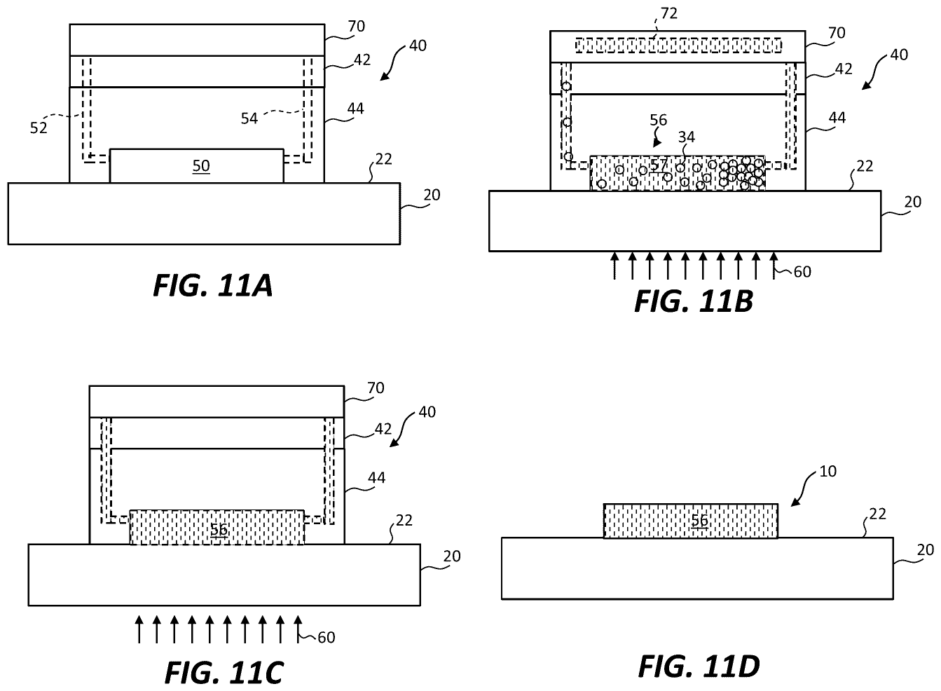

Inlet port 52 can be fed from a nanoparticle ink 56 supply. A pump and/or a dispenser 70 pumps or dispenses the nanoparticle ink 56 through the inlet port 52 and channel 50. Pump or dispenser 70 can provide pressure to infill nanoparticle inks in channels 50 at a greater speed and with reduced costs than is possible with capillary action alone. In FIGS. 11A-11D, a pump or dispenser 70 can provide nanoparticle inks 56 (e.g., comprising nanoparticles 12 in a dispersant or solvent 57) from pump reservoir 72 and ink reservoir 58 to inlet port 52 of micro-mold stamp 40 under pressure and a vacuum (or partial vacuum or reduced pressure) to outlet port 54 to draw nanoparticle ink 56 into and through channels 50. Micro-mold stamp 40 can comprise nanoparticle ink 56 reservoirs 58 for controlling the volume and flow rate of nanoparticle ink 56. Inlet port 52 and outlet port 54 can also serve as integrated ink reservoirs 58. In some embodiments the pressure driving nanoparticle ink 56 through channels 50 can be the capillary pressure caused by forces between nanoparticle ink 56 and the surface area of the micro-channels 50 in contact with nanoparticle ink 56.

Although FIGS. 9A-9D illustrate specific micro-molding machine structural schemes and compositions, any configuration and design can be utilized as appropriate depending on the specific requirements of the given application. Systems and methods of micro-molding fabrication processes that can be utilized in making electrical devices in accordance with various embodiments of the invention are discussed further below.

### Fabrication of Gas Sensors Using Micro-Molding Processes

Many embodiments provide micro-molding processes of electrical components and/or devices. Examples of electrical components and/devices include (but are not limited to) gas sensor elements, antennas, inductors. Micro-molding fabrication processes in accordance with several embodiments implement micro-molding machines. Many embodiments provide that micro-molding fabrication processes can include (but are not limited to) the following steps:
providing a substrate that has a substrate surface;
providing a stamp that has a mold layer with a support side and a channel side and a support layer disposed in contact with the support side;
providing a first ink including (but not limited to) nanoparticle ink and second ink including (but not limited to) nanoparticle ink;
disposing the mold layer in contact with the substrate surface;
pumping or dispensing the first nanoparticle ink through the first inlet port and into the first channel;
pumping or dispensing the second nanoparticle ink through the second inlet port and into the second channel;
curing the first nanoparticle ink in the first channel;
curing the second nanoparticle ink in the second channel;
removing the stamp to form a free-standing component on the substrate surface.

In several embodiments, the mold layer can include a first channel having a first form factor disposed on the channel side, a first inlet port connected to the first channel, and a first outlet port connected to the first channel. In some embodiments, the mold layer can include a second channel having a second form factor disposed on the channel side, a second inlet port connected to the second channel, and a second outlet port connected to the second channel. The mold stamps in accordance with certain embodiments can be made with materials including (but not limited to) polydimethylsiloxane, polymethyl methacrylate, and polyurethane. A number of embodiments provide that the first form factor of the first channel and the second form factor of the second channel can have the same or different form factors. In many embodiments, the support layer can be more rigid than the mold layer. Several embodiments provide that the channels can have a height in a direction into the mold layer from the channel side that is greater than a width of the channel on the channel side, or both. In some embodiments, features within the stamp can be positioned with respect to features on the substrate to ensure that micro-molded features are disposed at specific locations on the substrate, within a specified position tolerance including (but not limited to) 1 micron, or 10 microns. In certain embodiments, features within the stamp can be positioned with respect to features on the substrate using visual reference markers on the stamp and the substrate,

Certain embodiments provide that the first nanoparticle ink and the second nanoparticle ink can be the same or different nanoparticle inks. In some embodiments, curing the nanoparticle ink can form nano-porous fused nanoparticle electrical conductors. The nano-porous fused nanoparticle electrical conductors in accordance with several embodiments can have an electrical conductivity that changes in response to an ambient gas that is in contact with the nano-porous fused nanoparticle electrical conductors. The steps of curing the nanoparticle inks in accordance with several embodiments can be accelerated by heating the nanoparticle inks and/or by exposing the nanoparticle inks to electromagnetic radiation. In some embodiments, the nanoparticles can be sintered by heating the nanoparticles and/or by exposing the nanoparticles to electromagnetic radiation.

Many embodiments provide that providing inlet pressure to the inlet port and outlet pressure to the outlet port can pump the nanoparticle ink through the inlet port and into the channel, if the inlet pressure is greater than the outlet pressure. In several embodiments, pumping and/or dispensing the nanoparticle inks can cause the nanoparticle inks to flow through the channel and the flow of nanoparticle inks can be driven at least in part by capillary pressure in the channel. In certain embodiments, pumping and/or dispensing the nanoparticle inks can cause the nanoparticle inks to flow through the channel and the flow of nanoparticle inks can be driven by applying pressure to the inlet port and/or vacuum to the outlet port.

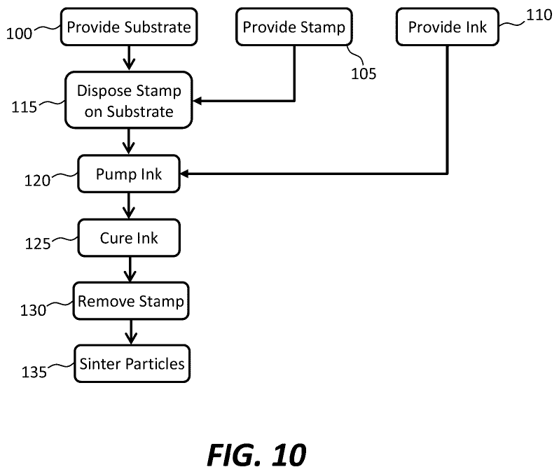

A process of fabricating a component using micro-molding processes in accordance with an embodiment of the invention is illustrated in FIG. 10. The fabrication process starts by providing a substrate for the component 100. A micro-molding stamp can be used to dispose the component 105. Mold layer of the micro-molding stamp can be disposed in contact with (for example in conformal contact with) the substrate surface of the substrate 115. A nanoparticle ink comprising nanoparticles in a liquid or gaseous solvent or dispersant can be provided 110. The nanoparticle ink comprising the nanoparticles can be pumped through the inlet port into channels 120. As nanoparticles move through the channels, solvent in nanoparticle ink can diffuse into the mold layer so that the nanoparticles become tightly packed in the channels. Complete wetting of the channels by the ink may be important to achieving the desired shape and facilitating fast extraction of the solvent, which can be achieved by careful tuning of solvent used and the surface energies of the stamp. The process can be accelerated by curing 125. The curing process in accordance with some embodiments includes (but not limited to) exposure the nanoparticle ink to heat, and/or to electromagnetic radiation. Examples of electromagnetic radiation include (but are not limited to) a xenon flash, infrared radiation, ultraviolet radiation, or laser radiation. During the curing processes, the solvent of the nanoparticle ink can be driven off from the nanoparticle ink and/or the mold layer. In some embodiments, the driven off solvent can be absorbed (at least in part) by the mold layer of the micro-molding stamp. Micro-molding stamp can be removed 130 to form a free-standing gas-sensor element on substrate surface of the substrate. Free-standing gas-sensor element can be formed not within a substrate or having supporting structures and/or walls. In several embodiments, nanoparticles can be sintered and/or fused to form gas-sensor elements 135. Sintering and/or fusing nanoparticles in accordance with certain embodiments can be accomplished by exposing nanoparticles to heat, UV radiation, laser radiation, or electromagnetic radiation. In a number of embodiments, the sintering process can be performed within a protective atmosphere including (but not limited to) nitrogen, helium, argon, hydrogen, carbon dioxide. Many embodiments provide that gas sensors can be constructed in a single layer and in a single series of steps, even when multiple gas-sensor elements have different form factors and/or comprise different nanoparticles. Although FIG. 10 illustrates specific steps of micro-molding fabrication process, any steps and methods can be utilized as appropriate depending on the specific requirements of the given application.

A successive cross section views of a high-aspect-ratio gas-sensor during the fabrication process in accordance with an embodiment is illustrated in FIGS. 11A-11D. In FIGS. 11A-11D, gas-sensor elements 10 can be constructed by providing a substrate a micro-mold stamp 40, and a nanoparticle ink 56 comprising nanoparticles 12 in a liquid or gaseous solvent or dispersant 57 provided. Mold layer 44 of micro-mold stamp is disposed in contact with (for example in conformal contact with) surface 22 (e.g., a surface of substrate 20 or insulating layer 96), as shown in FIG. 11A. As illustrated in FIG. 11B, nanoparticle ink 56 comprising nanoparticles 12 can be pumped through inlet port 52 into channels 50, for example by pump 70 from pump reservoir 72. Pumping can be provided at least in part by providing a pressure differential between inlet port(s) 52 and outlet port(s) 54. As nanoparticles 12 move through channels 50, solvent 57 in nanoparticle ink 56 diffuses into mold layer 44, drawing in more ink from the inlet- and outlet reservoirs 58, so that nanoparticles 12 become tightly packed in channels 50. This process continues until the average pore size within the structure is in the order of the pore size of the nanoparticles 12 in the ink and all solvent is extracted, finally leading to complete molding of the channel shape. Complete wetting of the channels by the ink may be important to achieving the desired shape and facilitating fast extraction of the solvent, which can be achieved by careful tuning of solvent used and the surface energies of the stamp.

In FIG. 11C, the curing process can be accelerated and/or enabled by, for example, exposing nano-particle ink 56 and/or mold layer 44 to heat and/or electromagnetic radiation 60 including (but not limited to) a xenon flash, ultraviolet radiation, or laser radiation, driving off solvent 57 which can be absorbed at least in part, by mold layer 44 of micro-mold stamp 40. Micro-mold stamp 40 is then removed to form a gas-sensor element 10 (optionally having a high-aspect ratio) on surface 22. Nanoparticles 12 can then be sintered or fused to form gas-sensor element 10 by exposing nanoparticles 12 to heat, UV radiation, or laser radiation. The sintering process can be performed within a protective atmosphere including (but not limited to) nitrogen, helium, argon, hydrogen, carbon dioxide. Gas-sensor element 10 can be free-standing, for example not formed within a substrate or having supporting structures or walls (except for underlying surface 22). Although FIGS. 11A-11D illustrate specific steps of micro-molding process of fabricating gas-sensors, any step and method can be utilized as appropriate depending on the specific requirements of the given application.

Systems of high-aspect-ratio antennas with high-aspect-ratio conductors that can be utilized in the design of antennas in accordance with various embodiments of the invention are discussed further below.

### Antennas

Antennas couple electrical voltages and currents to electromagnetic fields to enable communication or power transfer between spatially separated electronic devices. A great variety of antennas can be used for different applications, for example radios, television, WiFi, radar, and wireless power transfer (WPT). Antennas may include different sizes and configurations, operating at various frequencies, for example from 3 kHz to 300 GHz. Different bands of the electromagnetic spectrum are reserved for different applications, for example radio, television, and cellular telephony (smartphones). A complete antenna system operates by wirelessly coupling electrical energy from voltages and currents flowing in one set of electrical conductors (a “transmitter”), to voltages and currents induced in another set of electrical conductors (a “receiver”), via electromagnetic fields or inductive coupling.

Far-field (radiative) antenna systems tend to produce propagating electromagnetic waves even at great distances from the transmitter, regardless of whether or not a receiving antenna is present. In contrast, near-field (non-radiative) antenna systems produce strong evanescent fields in immediate proximity to the transmitter, are suitable for inductively coupling to a nearby receiver, but do not radiate power into propagating free-space electromagnetic modes. For antennas to operate efficiently in the far-field (radiative) regime, the physical extent of the antenna(s) is typically on the order of the wavelength of the signal being transmitted, or even much larger for directional antennas such as dish antennas. Far-field antennas can range from a few microns to hundreds of meters in extent, depending on frequency and antenna type. Near-field antennas can be dramatically smaller than the wavelength but tend to operate effectively only over distances of the same order of magnitude as the antenna size, and further tend to require low-loss conductors, careful resonance tuning, and precise alignment between transmitters and receivers. Many different antenna designs are in use, for example loop antennas, dipole antennas, microstrip antennas, monopole antennas, array antennas, and conical antennas.

A great variety of near-field antennas are used for different applications, for example near field communication (NFC) between electronic devices such as smartphones, radio frequency identification (RFID) tags and readers, wireless power transfer, data transfer in stacked ICs, and include many different sizes and configurations operating at various frequencies, for example from about 1 kHz to about 1 THz. Near-field antennas may be configured to receive and/or send data or power, and near-field devices may be powered by external power sources such as batteries, or directly by the power captured from the near-field, such as in the case of RFID.

Different bands of the electromagnetic spectrum are reserved for different applications, for example radio, television, and cellular telephony (smartphones). Near-field RFID systems typically operate in the low frequency range (LF, 125 KHz-134 KHz) or high frequency range (HF, 3 MHz-30 MHz) bands, such as 13.56 MHz RFID systems. However, operation of near-field RFID in the ultra-high frequency range (UHF, 300 MHz-3 GHz) band is also possible.

### Near-Field Coil Antennas

Antenna systems can comprise a single transmitting antenna and a single receiving antenna. Modern antenna systems can contain a multitude of transmitters and receivers each utilizing at least one individual antenna. Some antennas may function as both a transmitter and a receiver in such systems. Near-field antenna systems may rely on inductive coupling between two antennas, such as coil-type antennas, to transmit electrical signals and/or power. When an electrical signal is passed through one coil, an electromagnetic field can be created in its near-field region, which may induce electrical voltage or current within another coil proportional to the mutual inductance between the two coils. The mutual inductance can be maximized when the coils are oriented concentrically and as close together as possible, and when each coil itself has a greatest inductance, for example, by maximizing the number of windings within its footprint. Each turn of the traces around the internal space is known as a winding. To further improve the distance over which near-field antennas can operate, it might be useful to tune each coil for resonant operation at the operating frequency, and to minimize the resistive losses, to enable high-quality-factor (high-Q) operation. This may require precise control over the coil's dimensions.

Near-field antennas do not rely on radiating electromagnetic energy into the far field, such that they can be operated with very low transmission losses and are limited in principal by their own resistive losses. This makes near-field antennas favorable for applications such as RFID, short-distance communication, and wireless power transfer. Or more generally for any application in which establishing a direct electrical connection between to a device for data or power transmission is not possible or desirable, such as stacked integrated circuits (ICs).

Portable electronic devices are preferably small and light. Consequently, coil antennas and inductors in such portable electronic devices should be desirably small, but with closely spaced, low-resistance conductors to maintain performance. For many applications in microelectronics, it may be desirable to produce compact antenna coils with as high an inductance (as many windings as practicable) and as low a series resistance as possible for any given antenna footprint and conductor length. Techniques for making small electrical conductors in or on a substrate such as a printed circuit board and an integrated circuit, include subtractive techniques and additive techniques. Subtractive techniques can include photochemical machining, etching, laser cutting, and machining. Additive techniques can include masked physical deposition (e.g. vacuum deposition), electroplating, 3D printing, inkjet printing, and screen printing of conductive inks or pastes. However, inkjet printing and screen printing have limited resolution and limited reproducibility at the sub-millimeter scale with poorly controlled cross-sectional shape. Photochemical machining, consisting of patterning of an etch mask followed by chemical etching, as is used for PCB manufacturing, may result in isotropic undercutting of the masked conductor edges. The undercutting can limit the achievable form of the conductors as well as the gaps separating them, reducing conductor resolution. Electroplating onto a patterned metal seed-layer can improve conductivity of the traces but also suffers from reduced conductor resolution because the metal deposition proceeds in a nominally isotropic manner, again limiting the minimum gap between conductor traces. Finally, electrically conductive polymers as an alternative to metals can be limited in conductivity, for example several orders of magnitude below that of copper or silver.

In previous works, Ko et. al. described a method for patterning electrical conductors by coating a substrate with a nanoparticle solution and imprinting the coating with a structured polydimethylsiloxane mold. (See, e.g., Ko, et. al., Nano Letters, 2007, vol. 7, No. 7 pp. 1869-1877; the disclosure of which is incorporated herein by reference in its entirety.) Makihata and Pisano described printing with silver nanoparticle ink. (See, e.g., Makihata et al., The International Journal of Advanced Manufacturing Technology, 2019, 103, 1709-1719; the disclosure of which is incorporated herein by reference in its entirety.)

Many embodiments provide design and fabrication methods of compact, high-Q antenna structures and/or inductor coils to improve the performance of various electrical circuits and wireless devices. Several embodiments provide compact antenna coils with high inductance and low series resistance by fabricating coils of highly conductive material, with closely spaced, high aspect-ratio traces.

High-aspect-ratio coils in accordance with many embodiments can be applied in the fabrication of high-Q, low-loss air-core inductors. The high-Q low-loss air-core inductors play important roles in high-frequency electronic circuit design. Many embodiments implement high-aspect-ratio coil structures as inductors in areas including (but not limited to): switch mode power supplies, radio frequency (RF) band-pass, high-pass, and low-pass filters, low-loss transformers, inductive angle and position sensors, and LC or RLC resonators. The printed inductors and/or coils in accordance with several embodiments can be integrated as discrete components as part of a larger distributed element network or microstrip containing multiple passive components. In such embodiments, the high accuracy of the printed inductor/coils can provide benefits including (but not limited to): more accurate tuning of the resonance frequency, smaller footprint, sub-quarter wavelength filtering, higher power coupling efficiency.

Systems of high-aspect-ratio antennas with high-aspect-ratio conductors that can be utilized in the design of antennas in accordance with various embodiments of the invention are discussed further below.

### High-Aspect-Ratio Antennas

A common type of near-field antenna is the inductive coil, comprising a helical or spiral arrangement of conductive electrical material. Such electrical conductors can be wires with various cross-sectional profiles, for example cylindrical wires, rectangular wires, or planar electrical conductors. Many embodiments implement high-aspect-ratio electrical conductors as the electrical conductors. These wires and/or traces in accordance with embodiments can be arranged in configurations including (but not limited to) planar rectangular, circular, or hexagonal spiral on a substrate to form a coil. The coil can have external dimensions between about 1×1 μm² and about 1×1 m². In some embodiments, the coil can contain an internal space which is not occupied by coiled conductors, such as an air-core inductor. In several embodiments, the internal space of the coil may be occupied by a magnetic core to increase the inductance. Coils can be extended into the normal direction with respect to the substrate in accordance with certain embodiments, so that the conductor has an increased aspect ratio. In some embodiments, several coils can be stacked to increase the inductance of the coil. In a number of embodiments, multiple coaxially located coils can be placed around the same axis. These coils can be located same plane or substrate or be placed at subsequent planes or substrates along the same axis. The design of the coil can be symmetrical or asymmetrical.

The quality and bandwidth of the signal transmission between two near-field antennas depend on their mutual inductance, the magnitude of the current passed through the transmitting antenna, and the frequencies at which the coils are driven. The mutual inductance can be determined by the physical separation and orientation between the two antennas, as well as their respective self-inductances. The radius of the antennas should be tuned to the distance at which the signal is expected to be received. For example, a pair of coil antennas with external dimensions of about 10 mm can give a best transmission when the separation distance is approximately 12 mm. The mutual inductance increases with the number of turns in each of the coils. The antenna should have a low electrical resistance. Higher resistance may result in attenuation of fields and signal strength, as well as undesired power dissipation and heating in the device.

Many embodiments provide high-aspect-ratio electrical conductors arranged in a variety of configurations for high-performance inductors and antennas including (but not limited to) near-field antennas. In some embodiments, antennas with high-aspect-ratio conductors can be fabricated as free-standing structures formed or deposited on a substrate. The high-aspect-ratio conductors in accordance with some embodiments can be constructed from nanoparticle inks cured in channels disposed in stamps applied onto a substrate surface. Several embodiments provide that these processes enable antennas and inductors to be made with dimensions suitable for small and portable electronic devices. In certain embodiments, the antennas and conductors have dimensions in the range from about 1 μm to about 100 μm.

Many embodiments provide that the substrate of the high-aspect-ratio antennas can be any suitable substrate including (but not limited to) glass, polymers, Kapton (polyimide), PET, PMMA, Teflon (PTFE), ETFE, ceramics, low temperature co-fired ceramics (LTCC), semiconductors, Si, SiO₂, Si₃N₄, SiC, GaAs, GaInP, InP, quartz, metals, paper, and/or sapphire. In several embodiments, the substrate can be a printed-circuit board (PCB) substrate including (but not limited to) FR2 or FR4. In some embodiments, the substrate can be rigid, flexible, or planar. A number of embodiments provide that the substrates can be found in the display, integrated circuit, electronics assembly, or circuit board industries. In some embodiments the substrate may contain CMOS and/or MEMS devices, integrated circuits, microprocessors, microcontrollers, angle measurement circuitry, RF circuits, and transceivers.

In many embodiments, the high-aspect-ratio antennas can be made with particles including (but not limited to) electrically conductive particles, metallic nanoparticles, electrically non-conductive (dielectric) particles, or semi-conducting particles. Examples of nanoparticles include (but are not limited to) silver, copper, gold, nickel nanoparticles, or any combinations thereof. In some embodiments, nanoparticles can be sintered. In certain embodiments, nanoparticles can be coated by a conductor. In a number of embodiments, nanoparticles can be coated by a thin metal coating by electroplating. Electroplating can provide a metallic coating over a surface but can also deposit the coating material on the substrate surface that may reduce the spatial resolution of structures formed on the substrate surface. In several embodiments, nanoparticles are not electroplated. Examples of semi-conducting particles include (but are not limited to) metal oxides. In several embodiments, the particles can be provided as a suspension in liquid solvent including (but not limited to) aqueous dispersants, organic solvents, isopropanol, ethanol, toluene, ethylene glycol, propylene glycol, diethylene glycol, triethylene glycol, diethylene glycol monomethyl ether, or triethylene glycol monomethyl ether. Nanoparticles in accordance with certain embodiments can have diameters in the range from about 1 nm to about 5 μm. Some embodiments provide that suitable inks can have viscosities in a range from about 0.3 centipoise to about 3000 centipoises. In some embodiments, nanoparticles can include different nanoparticles made of different conductive and/or non-conductive materials. In several embodiments, nanoparticles can be distributed isotropically or anisotropically in antenna.

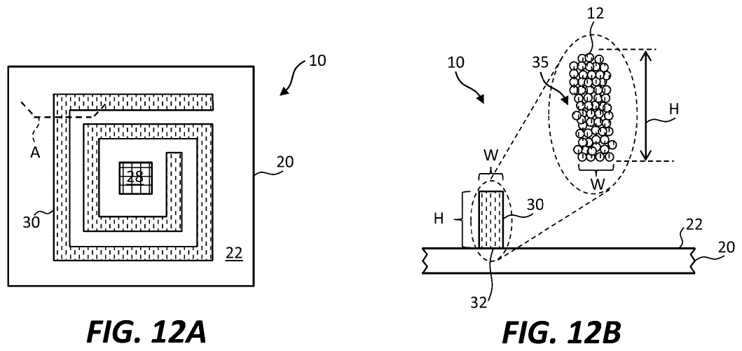

Many embodiments provide high-aspect-ratio antenna structures. In several embodiments, antennas can form a coil on a substrate. A plan view of a high-aspect-ratio antenna in accordance with an embodiment of the invention is illustrated in FIG. 12A. A high-aspect-ratio antenna structure 10 includes a substrate 20 having a substrate surface 22. An antenna 30 is disposed on the substrate surface 22. The antenna 30 can be in planar rectangular, circular, or hexagonal spiral on the substrate surface 22 to form a coil.

A cross section view of a high-aspect-ratio antenna taken along across section line A of FIG. 12A in accordance with an embodiment of the invention is illustrated n FIG. 12B. A high-aspect-ratio antenna structure 10 includes a substrate 20 having a substrate surface 22. An antenna 30 is disposed on the substrate surface 22. The antenna 30 can have a rectangular cross section, or any other desirable cross section including (but not limited to) triangular, quadrilateral, or with a curved surface. The antenna 30 can be electrically connected to a circuit (not shown) that operates or responds to antenna 30. The antenna 30 can be made of fused nanoparticles 12. The electrical conductor of antenna 30 has a base 32 having a conductor width W in contact with the substrate surface 22 and a conductor height H in a direction extending away from the substrate surface 22. The antenna 30 can be free-standing on the substrate surface 22 without support other than support from the base 32 on the substrate 20. The conductor height H can be greater than the conductor width W. In some embodiments, the antenna 30 can have an exposed conductor surface 35 of fused nanoparticles 12 on at least one point along the conductor. In several embodiments, the conductor surface 35 can be coated with a conducting material disposed on the fused nanoparticles 12. Exposed conductor surface 35 can be the outside edge or the surface of antenna 30, optionally excluding base 32. In various embodiments, the electrical conductor of antenna 30 can vary in size, height, width, aspect ratio, composition, and density over the length of the electrical conductor on substrate 20.

In many embodiments, the base of antenna disposed on substrate surface can have a conductor width W of less than 50 microns. Several embodiments provide that the conductor width W of less than 25 microns, of less than 10 microns, of less than 5 microns, or of less than 2 microns. Conductor height H of antenna extending away from substrate surface in accordance with some embodiments can be greater than 5 microns. In certain embodiments, the conductor height H can be greater than 10 microns, greater than 20 microns, greater than 50 microns, or greater than 100 microns. In many embodiments, antennas have an aspect ratio (a ratio of conductor height H to conductor width W) of greater than 1. In some embodiments, the aspect ratio of the antenna can be greater than 2.8, greater than 5, greater than 10, or greater than 20. Certain embodiments provide that antenna with the aspect ratio of greater than 2.8 can have a conductor width W of about 2.5 microns and a conductor height of about 7 microns.

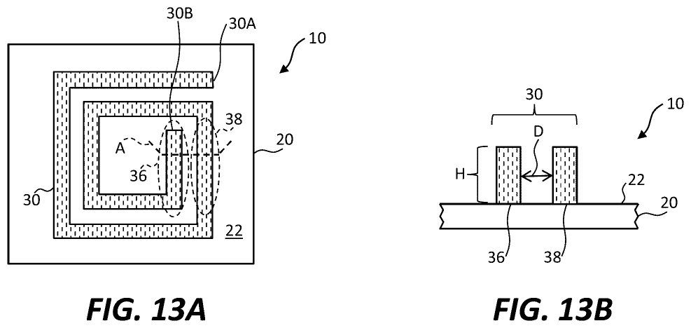

Many embodiments provide coil antennas incorporating high-aspect-ratio electrical conductors. In several embodiments, coil antennas have a conductor length L that extends from one end of the coil antenna to the other end of the coil antenna. The coil antennas in accordance with some embodiments have a separation distance D between the windings of antenna. In many embodiments, the high-aspect-ratio antenna structures can provide a greater number of windings N of the antenna in a reduced area and/or volume, enabling improved sensitivity to electromagnetic radiation within a range of frequencies. Examples of the frequency range include (but are not limited to) frequencies less than 867 MHz. Such sensitivity can be useful in small form factors in portable electronic devices. A plan view of a coil antenna in accordance with an embodiment of the invention is illustrated in FIG. 13A. In FIG. 13A, a high-aspect-ratio coil antenna 10 includes a coil antenna 30 disposed on substrate surface 22 of a substrate 20. The antenna 30 has a first portion 36 on the substrate 20 that is adjacent to a second portion 38 on the substrate 20. The first portion 36 and the second portion 38 are spaced apart by a distance D over the substrate surface 22.

In FIG. 13A, conductor length L of antenna 30 extends from antenna first end to antenna second end 30B with a small separation distance D between the windings of antenna 30 (corresponding to first and second portions 36, 38 of antenna 30). Thus, high-aspect-ratio antenna structures 10 can provide a greater number of windings N of antenna 30 over substrate 20 in a reduced area or volume enabling improved sensitivity to electromagnetic radiation within a desired range of frequencies.

A cross section view of the high-aspect-ratio coil antenna taken along the cross-section line A of FIG. 13A in accordance with an embodiment of the invention is illustrated in FIG. 13B. The coil antenna 30 is disposed on substrate surface 22 of a substrate 20. The first portion 36 and the second portion 38 are spaced apart by a distance D over the substrate surface 22. Distance D can be no greater than conductor height H. In some embodiments, first portion 36 is separated from second portion 38 by a distance D less than 50 microns. In several embodiments, the distance D between the first portion 36 and the second portion 38 is less than 25 microns, less than 20 microns, less than 15 microns, less than 10 microns, and less than 5 microns. Many embodiments provide that the windings of antenna 30 are spaced closely together, enabling a large conductor length L of antenna 30 in a small area over substrate 20. In some embodiments, antenna 30 (e.g., a coil) has a single turn. In several embodiments, the coil has multiple turns with one or more adjacent first and second portions 36 and 38, as shown in FIG. 13A. In certain embodiments, antenna 30 has straight line segments joined together at discontinuous corners. In a number of embodiments, the corners of the electrical conductor in antenna 30 are orthogonal (90 degree) corners. In some embodiments, the corners are not orthogonal. Examples of non-orthogonal angels include (but are not limited to): 60 degrees, 120 degrees, or 150 degrees. Several embodiments provide that antenna 30 has straight line segments. According to some embodiments, antenna 30 has curved segments or is entirely curved.

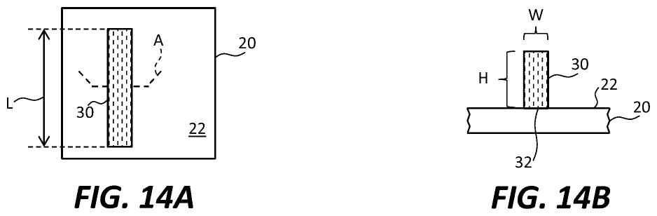

A plan view of an antenna with antenna length L in accordance with an embodiment of the invention is illustrated in FIG. 14A. An antenna 30 has an antenna length L from a first end of the antenna to a second end of the antenna is disposed on substrate surface 22 of a substrate 20. A cross section view of the antenna taken along the cross-section line A of FIG. 14A in accordance with an embodiment of the invention is illustrated in FIG. 14B. The antenna 30 has an antenna base 32, an antenna width W, and an antenna height H.

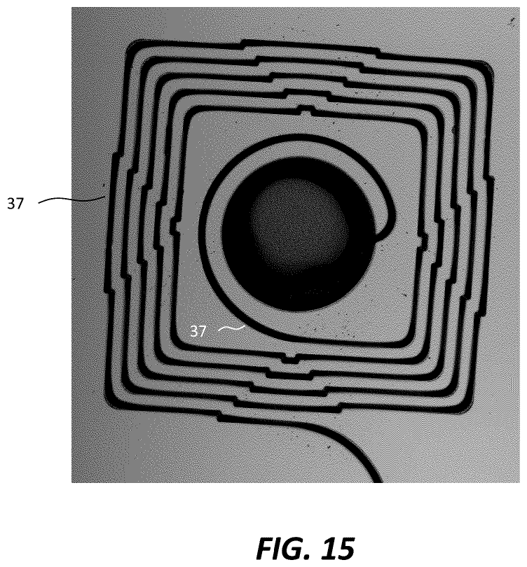

Several embodiments provide that coil antennas can have segments incorporating thermal strain reliefs to prevent buckling of the segments during (rapid) temperature changes. Rapid temperature changes can occur during sintering or operation of the antenna. The thermal strain reliefs in accordance with some embodiments can divide these segments into multiple shorter segments to prevent buckling. A coil antenna incorporating thermal strain reliefs in accordance with an embodiment of the invention is illustrated in FIG. 15. The thermal strain reliefs 37 are incorporated in segments of coil antennas to prevent buckling of the segments during (rapid) temperature changes by dividing these segments into multiple shorter segments. Although FIGS. 12-15 illustrate specific high-aspect-ratio antenna structural schemes and compositions, any configuration and design can be utilized as appropriate depending on the specific requirements of the given application.

Systems and methods for making high-aspect-ratio antennas with high-aspect-ratio conductors using micro-molding processes that can be utilized in the design and/fabrication of antennas in accordance with various embodiments of the invention are discussed further below.

### Fabrication of High-Aspect-Ratio Antennas Using Micro-Molding Processes

Many embodiments provide that high-aspect-ratio microstrip antennas can be used for high frequency (frequency higher than about 100 MHz) applications including (but not limited to) millimeter wave antennas, and microwave antennas. Typically, microstrip antennas are manufactured by creating an etch mask using photolithography and subsequently etching the metal. This is a multi-step processes which can limit the choice of substrates on which such circuits can be processed. Typically, the metal etch step has to occur early in the production process to avoid damaging or degrading complex structures or devices such as ICs present on the substrate. Metal etching can also limit the feasible aspect ratio of antenna conductors, since the conductor thickness has to be smaller than the feature spacing. Moreover, common metal etching technologies can be limited by the isotropic nature of the etching process, which may limit the form accuracy that can be achieved for a high-aspect-ratio structure. On the other hand, evaporation of metal onto a masked substrate in vacuum, followed by lift-off step to remove the mask, to form high-aspect-ratio structures would waste a large portion of the material which cannot be recovered, whereas evaporation of a thin film of metal followed by electrochemical deposition of metal can limit feature spacing and fidelity.

Many embodiments provide that a high-aspect-ratio antenna structure can include a plurality of antennas including (but not limited to) coil antennas disposed on a substrate. The plurality of antennas in accordance with several embodiments can form a phased-array antenna.

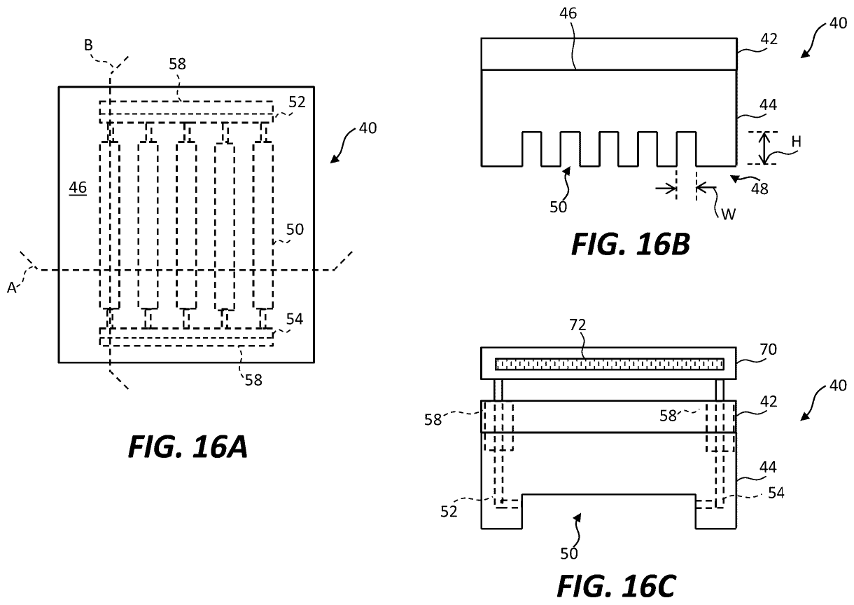

Several embodiments provide high-aspect-ratio antennas can be constructed using a micro-mold stamp. A plan view of a micro-mold stamp in accordance with an embodiment of the invention is illustrated in FIG. 16A. A cross section view of the micro-mold stamp taken across the cross-section line A of FIG. 16A is illustrated in FIG. 16B. A cross section view of the micro-mold stamp taken across the cross-section line B of FIG. 16A is illustrated in FIG. 16C. Micro-mold stamp 40 can comprise a mold layer 44 having a support side 46 and a channel side 48. A support layer 42 is disposed in contact with support side 46. Support layer 42 can be more rigid than mold layer 44 to provide dimensional stability to mold layer 44 and enable improved resolution for structures formed by micro-mold stamp 40. Mold layer 44 can comprise at least one channel 50 disposed on the channel side 48 in mold layer 44. An inlet port 52 is connected to the channel 50, and an outlet port 54 is connected to the channel 50. Channel 50 has a height in a direction into mold layer 44 away from channel side 48 toward support side 46 (corresponding to conductor height H) that is greater than a width of the channel 50 on the channel side 48 (corresponding to conductor width W). In some embodiments, inlet and outlet ports 52 and/or 54 can extend to channel side 48 surface of the mold layer 44. Inlet ports 52 provide a path for nanoparticle inks 56 to enter channels 50 and outlet ports 54 provide a path for nanoparticle inks to be drawn into or out of channels 50. Mold layer 44 can comprise an elastomeric material including (but not limited to) polydimethylsiloxane, cast and cured on a photolithographically defined master including (but not limited to) a silicon master, a quartz master, or a glass master. In several embodiments, mold layer 44 can be reinforced by incorporation of nanoparticles into the elastomeric material, or by the inclusion of a fiber mesh composed of including (but not limited to) glass, steel, carbon, or nylon. Support layer 42 can comprise a more rigid material including (but not limited to) glass, than mold layer 44, and can be thinner than mold layer 44.

In FIG. 16C, a pump and/or a dispenser 70 can provide nanoparticle inks from a pump reservoir 72 to inlet port 52 of the micro-mold stamp 40 under pressure and a vacuum (or partial vacuum or reduced pressure) to outlet port 54 to draw nanoparticle ink 56 into and through channels 50. Micro-mold stamp 40 can comprise nanoparticle ink reservoirs 58 for controlling the volume and flow rate of nanoparticle ink 56. Inlet port 52 and outlet port 54 can also serve as integrated ink reservoirs 58. In some embodiments, the pressure driving the ink through the channels can be the capillary pressure caused by forces between the nanoparticle ink 56 and the surface area of the microchannels 50 in contact with the ink.

A process of fabricating high-aspect-ratio antennas in accordance with an embodiment of the invention is illustrated in FIG. 10. The fabrication process starts by providing a substrate for the high-aspect-ratio antennas 100. A micro-mold stamp can be used to dispose the antennas 105. Mold layer of the micro-mold stamp can be disposed in contact with (for example in conformal contact with) the substrate surface of the substrate 115. A nanoparticle ink comprising nanoparticles in a liquid or gaseous solvent or dispersant can be provided 110. The nanoparticle ink comprising the nanoparticles can be pumped through the inlet port into channels 120. As nanoparticles move through the channels, solvent in nanoparticle ink can diffuse into the mold layer so that the nanoparticles become tightly packed in the channels. The process can be accelerated by curing 125. The curing process in accordance with some embodiments includes (but not limited to) exposure the nanoparticle ink to heat, and/or to electromagnetic radiation. Examples of electromagnetic radiation include (but are not limited to) a xenon flash, infrared radiation, ultraviolet radiation, or laser radiation. During the curing processes, the solvent of the nanoparticle ink can be driven off from the nanoparticle ink and/or the mold layer. In some embodiments, the driven off solvent can be absorbed (at least in part) by the mold layer of the micro-mold stamp. In certain embodiments, the driven off solvent can diffuse through the mold layer into the environment surrounding the stamp. Examples of the environment surrounding the stamp include (but are not limited to) air, vacuum, or inert gasses including (but not limited to) nitrogen and argon. Micro-mold stamp can be removed 130 to form a free-standing antenna with high aspect ratio conductors on substrate surface of the substrate. The free-standing antenna can then be sintered 135 by exposing nanoparticles to heat, UV radiation, or laser radiation. Many embodiments provide that antennas can be constructed in a single layer and in a single series of steps. The fabrication processes of the antennas in accordance with several embodiments avoid repeated deposition and patterning steps.

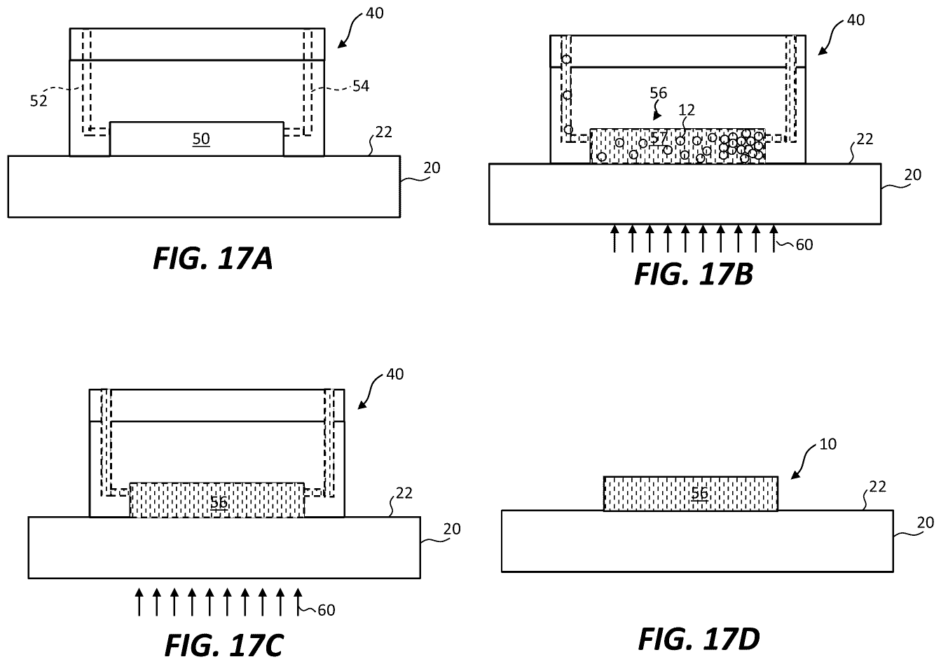

A successive cross section views of a high-aspect-ratio antenna during the fabrication process in accordance with an embodiment is illustrated in FIGS. 17A-17D. High-aspect-ratio antenna structures in accordance with some embodiments can be constructed by providing a substrate 20, a micro-mold stamp 40, as shown in FIG. 17A. The mold layer 44 of micro-mold stamp 40 is disposed in contact with (for example in conformal contact with) substrate surface 22 of substrate 20, as shown in FIG. 17A. A nanoparticle ink 56 comprising nanoparticles 12 in a liquid or gaseous solvent or dispersant 57 can be pumped through inlet port 52 into channels 50, for example by pump, as shown in FIG. 17B. As nanoparticles 12 move through channels 50, solvent in nanoparticle ink 56 diffuses into the mold layer 44 so that nanoparticles 12 become tightly packed in channels 50. In FIG. 17C, this process can be accelerated and/or enabled by the exposure to heat and/or electromagnetic radiation 60 (e.g., a xenon flash, infrared radiation, ultraviolet radiation, or laser radiation) of nanoparticle ink 56 and/or mold layer 44. The process can drive off solvent 57 which can be absorbed at least in part, by mold layer 44 of micro-mold stamp 40, or which can diffuse through the mold layer into the environment surrounding the stamp. Micro-mold stamp 40 can then be removed to form a free-standing antenna with high aspect ratio conductors 30 on substrate surface 22 of substrate 20 in FIG. 17D. The free-standing antenna 30 can then be sintered by exposing nanoparticles 12 to heat, UV radiation, or laser radiation. Antenna 30 can be constructed in a single layer and in a single series of steps.

Although FIGS. 16A-16C and FIGS. 17A-17D illustrate specific steps of micro-molding fabrication process of high-aspect-ratio antennas, any step and method can be utilized as appropriate depending on the specific requirements of the given application. Systems and methods for integrating high-aspect-ratio antennas with circuit components in accordance with various embodiments of the invention are discussed further below.

### Integration of High-Aspect-Ratio Antennas

Many embodiments provide high-aspect-ratio antennas can be integrated into an electronic circuit including (but not limited to) a tuned antenna system. In several embodiments, components including (but not limited to) circuits, integrated circuits (ICs), resistors, and capacitors can be incorporated into the antenna systems. The added components in accordance with some embodiments can be placed inside and/or outside the coil. In certain embodiments, the components can be placed within a different circuit plane.

To receive signals from the coil antennas, both ends of the spiral conductor trace may need to be electrically connected to an external circuit, requiring out-of-plane circuit connections to one or both ends of the antenna spiral. Many embodiments provide antenna system designs to enable excitation and/or receival signals from the coil antennas. Several embodiments incorporate a conductive trace fabricated either above or below the coil. The conductive trace in accordance with certain embodiments can connect an inner-most coil to a coplanar region outside of the coil. In a number of embodiments, the conductive trace can connect an outer-most coil to a coplanar region within the coil. Many embodiments provide that the electrical connection can be made either above or under the traces or wires that form the coil. In several embodiments, the electrical connections can be made by wire bonding, or by depositing a separate conductor on top of or below the coil. In some embodiments, the electrical connections can be made together with an electrical insulation (dielectric) layer to avoid shorting between the coil loops of the high-aspect-ratio antennas. Many embodiments provide that antennas with high-aspect-ratio conductors can be disposed on at least one component including (but not limited to) electrical conductor, dielectric, other structure, other high-aspect-ratio structure, layer, MEMS device, CMOS device, or structured layer. Several embodiments provide that antennas can be electrically connected to circuits including (but not limited to) integrated circuit controllers, circuits responsive to signals provided through high-aspect-ratio antenna.

In many embodiments, high-aspect-ratio antenna can comprise an antenna portion disposed on a structure and an antenna portion disposed on a different structure. In several embodiments, the two ends of high-aspect-ratio antennas are connected to the two different portions of the antenna. In some embodiments, one portion of the antennas can be disposed over and electrical conductor, and the other portion of the antennas can be disposed over an electrically insulating dielectric. Such structures can enable electrical conductors to electrically connect to one end but not to the other end of the antenna. The independent electrical connections in accordance with several embodiments can be made to different ends of the antenna. In some embodiments, the independent electrical connections can be made between the antennas and electrical circuits, such as integrated circuits in the interior of or exterior to the coil antennas. The independent electrical connections in accordance with certain embodiments can avoid undesired electrical connections to other portions of antenna.

In some embodiments, high-aspect-ratio antennas can be coated with a material including (but not limited to): an encapsulant, a dielectric encapsulant, or a metal coating. Examples of encapsulants can include (but are not limited to) polymers including (but not limited to) curable polymers, epoxy, polydimethylsiloxane, polyurethane, low temperature cofired ceramic (LTCC) sheets. The coating in accordance with certain embodiments can protect antennas from environmental contaminants. In some embodiments, the encapsulant coating layers can form a more mechanically robust structure of the antennas. In several embodiments, the encapsulant layers can enhance the electromagnetic properties of antennas, such as by improving its conductivity. The encapsulant layer in accordance with embodiments can planarize antenna or form a conformal coating over antennas.

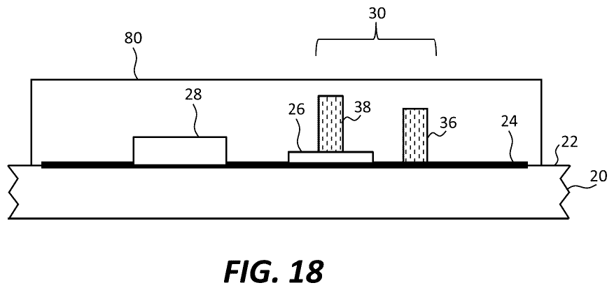

An antenna system in accordance with an embodiment of the invention is illustrated in FIG. 18. In some embodiments, the high-aspect-ratio antenna 30 can be placed on substrate surface 22 of a substrate 20. In several embodiments, antenna 30 with high-aspect-ratio conductors can be disposed over a structure 26 on substrate 20. In certain embodiments, antenna 30 can be disposed on an electrically conductive substrate contact 24 that provides an electrical connection to antenna 30. Substrate contact 24 can extend over substrate 20, or cover only selected area. First and second portions 36, 38 of antenna 30 can be disposed over different structures on substrate 20, for example an electrical conductor 24 and an electrically insulating dielectric 26. Such structures can enable electrical conductors to electrically connect a first portion 36 of antenna 30 but not to the second portion 38 of antenna 30, so that independent electrical connections can be made to a first end 30A (shown in FIG. 13A) of a coil antenna 30 and to a second end 30B (shown in FIG. 13A) of coil antenna 30, or to electrical circuits 28 in the interior of coil antenna 30 or exterior to coil antenna 30 without undesired electrical connections to other portions of antenna 30. Thus, high-aspect-ratio antenna 30 can comprise a first antenna portion 36 disposed on a first structure (e.g., substrate contact 24) and a second antenna portion 38 is disposed on a second structure (e.g., dielectric 26) different from the first structure. The antenna 30 can be coated with an encapsulant for protection.

In many embodiments, high-aspect-ratio antennas can be a multi-layer antenna. Each antenna layer in accordance with some embodiments can be separated by an insulator from adjacent layers and connected through electrical vias. Several embodiments provide that the conductive pathway between the outer and inner regions of the coil antenna can be made by a second coil of opposite chirality. The second coil of opposite chirality in accordance with certain embodiments can be placed concentric to the first coil, above or below the first coil. Some embodiments provide that the first and second coil antennas can be electrically isolated from each other by insulators except points of connection at the innermost or outermost extent of the coils. In several embodiments, vias can electrically connect electrical conductors in one antenna layer with electrical conductors in another antenna layer. In such embodiments, the inductance of the multi-layer coil structure can be greatly improved as compared to the single-layer coil, while providing a coplanar point at which to connect the coil to external circuitry.

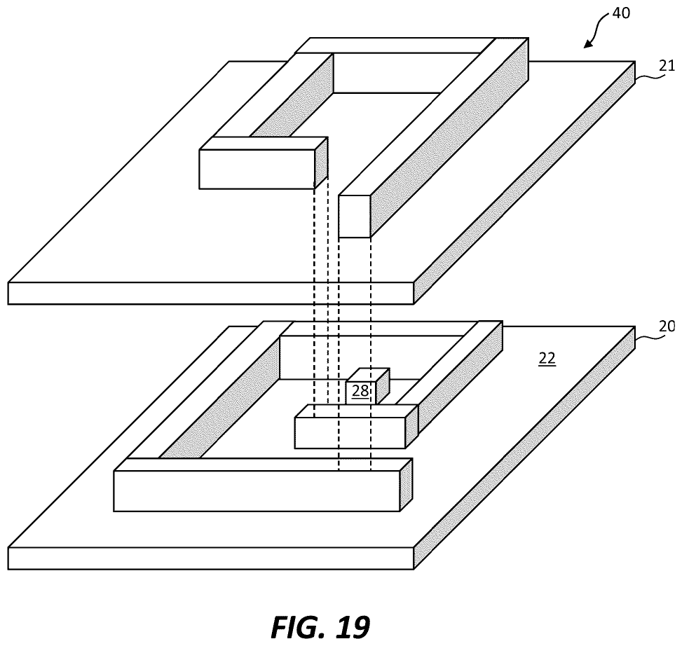

A multi-layer high-aspect-ratio antenna in accordance with an embodiment of the invention is illustrated in the exploded perspective in FIG. 19. A conductive pathway between the outer and inner regions of the coil can be made by a second coil of opposite chirality, concentric to the first coil, placed above (as shown in FIG. 19) or below the first coil and electrically isolated from it by insulators 21 at all areas except a single point of connection at the innermost or outermost extent of the coils. Vias, indicated by dashed lines in FIG. 19, can electrically connect electrical conductors in one antenna layer with electrical conductors in another antenna layer. In this way the inductance of the multi-layer coil structure can be greatly improved as compared to the single-layer coil, while providing a coplanar point at which to connect the coil to external circuitry. Although FIG. 18 and FIG. 19 illustrate implementing specific elements and components into high-aspect-ratio antennas, any configuration and design can be utilized as appropriate depending on the specific requirements of the given application.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

1. A micro-molded gas sensor, comprising:
at least one gas-sensor element, wherein the at least one gas-sensor element comprising a nano-porous electrical conductor, wherein the nano-porous electrical conductor comprising fused nanoparticles;
at least one first electrode electrically connected to a first end of the at least one gas-sensor element; and
at least one second electrode electrically connected to a second end of the at least one gas-sensor element;
wherein the at least one gas-sensor element has a corresponding first electrode and second electrode pair; and
wherein an electrical characteristic of the at least one gas-sensor element measured by the at least one first electrode and the at least one second electrode changes in response to an ambient gas in contact with the nano-porous electrical conductor.

2. The micro-molded gas sensor of claim 1, further comprising a first gas-sensor element and a second gas-sensor element, wherein the first gas-sensor element comprises a first nanoparticle composition, and the second gas-sensor element comprises a second nanoparticle composition different from the first nanoparticle composition.

3. The micro-molded gas sensor of claim 1, further comprising a first gas-sensor element and a second gas-sensor element, wherein the first gas-sensor element has a first form factor, and the second gas-sensor element has a second form factor different from the first form factor.

4. The micro-molded gas sensor of claim 1, further comprising a micro-heater to heat the at least one gas-sensor element.

5. The micro-molded gas sensor of claim 4, wherein the micro-heater comprises a plurality of micro-heater segments that are individually controllable to provide a different temperature in each of the plurality of micro-heater segments simultaneously.

6. The micro-molded gas sensor of claim 1, further comprising a sensor controller electrically connected to the at least one first electrode and electrically connected to the at least one second electrode, wherein the sensor controller is operable to provide electrical current to, and measure the resistivity of, the at least one gas-sensor element.

7. The micro-molded gas sensor of claim 1, further comprising:
a substrate;
a micro-heater disposed on the substrate; and
an electrically insulating layer disposed on the micro-heater,
wherein the at least one first electrode and the at least one second electrode are disposed on the electrically insulating layer and the at least one gas-sensor element is disposed on the corresponding first electrode and second electrode pair.

8. The micro-molded gas sensor of claim 7, wherein the at least one gas-sensor element does not extend beyond the micro-heater.

9. The micro-molded gas sensor of claim 7, wherein the substrate incorporates at least one membrane, wherein the membrane has a thickness less than about 1 micron.

10. The micro-molded gas sensor of claim 1, wherein the nanoparticles are selected from the group consisting of metal nanoparticles, metal-oxide nanoparticles, and doped metal-oxide nanoparticles.

11. The micro-molded gas sensor of claim 10, wherein the metal-oxide nanoparticles are one or more of: SnO₂, TiO₂, WO₃, ZnO, In₂O₃, Cd:ZnO, CrO₃, and V₂O₅.

12. The micro-molded gas sensor of claim 11, wherein the metal-oxide nanoparticles are doped with Al, Pt, Pd, Au, Ag, Ti, Cu, Fe, Sb, Mo, Ce, Mn, Rh₂O₃, or carbon nanotubes.

13. The micro-molded gas sensor of claim 1, wherein the at least one gas-sensor element has a height in the range of about 1 μm to about 20 μm, and a width in the range of about 1 μm to about 50 μm.

14. The micro-molded gas sensor of claim 1, wherein the at least one gas-sensor element has a surface roughness of less than about 100 nm RMS.

15. The micro-molded gas sensor of claim 1, wherein the ratio between an element height of the at least one gas-sensor element and an element width of the at least one gas-sensor element is no less than 2.

16. The micro-molded gas sensor of claim 1, wherein the ratio between an element height of the at least one gas-sensor element and an element width of the at least one gas-sensor element is no greater than 0.5.

17. The micro-molded gas sensor of claim 1, wherein the ratio between a spacing between at least two adjacent gas sensor elements and an element width of the at least one gas-sensor element is no more than 4.

18. The micro-molded gas sensor of claim 1, further comprising at least one force electrode that injects current or voltage into the at least one gas-sensor element, and at least one sense electrode that measures a change in an electrical characteristic.
